(19) United States
(12) Patent Application Publication
(10) Pub. No.: US 2019/0224166 A1
(43) Pub. Date: Jul. 25, 2019

(54) Compositions and Methods for Treating Muscle Loss

(71) Applicant: Augusta University Research Institute, Inc., Augusta, GA (US)

(72) Inventors: Mark Hamrick, Augusta, GA (US); Helen Kaiser, Augusta, GA (US)

(21) Appl. No.: 16/253,431

(22) Filed: Jan. 22, 2019

Related U.S. Application Data
(60) Provisional application No. 62/620,173, filed on Jan. 22, 2018.

(51) Int. Cl. A61K 31/405; A61K 9/00; A61P 21/00

(52) U.S. Cl. CPC A61K 31/405; A61P 21/00; A61K 9/0053

(57) ABSTRACT

Compositions of indoleamine 2,3-dioxygenase (“IDO”) inhibitors and methods of use thereof are provided. The disclosed compositions may be used to inhibit or reduce kynurenine production in the blood, treat or prevent muscle loss, increase or maintain muscle mass, muscle strength and/or muscle function, and treat or prevent sarcopenia.

### CROSS-REFERENCE TO RELATED APPLICATIONS

This application claims benefit of and priority to U.S. Provisional Patent Application No. 62/620,173 filed on Jan. 22, 2018, which is incorporated by reference in its entirety.

### STATEMENT REGARDING FEDERALLY SPONSORED RESEARCH

This invention was made with government support under AG036675 awarded by the National Institutes of Health. The government has certain rights in the invention.

### TECHNICAL FIELD OF THE INVENTION

The invention generally relates to compositions including indoleamine 2,3-dioxygenase (“IDO”) inhibitors and their use to treat or prevent muscle loss and sarcopenia

### BACKGROUND OF THE INVENTION

Aging is associated with a marked decrease in muscle fiber size, muscle mass, and muscle power. This condition is referred to as sarcopenia. Sarcopenia has been defined as an age related, involuntary loss of skeletal muscle mass and strength (Walston, J. Curr Opin Rheumatol. 24(6): 623-627 (2012)). Sarcopenia does not require an underlying disease for manifestation. Loss of muscle mass and power with age in the form of sarcopenia is associated with functional decline and a loss of independence in older adults. The etiology of sarcopenia includes decreased physical activity and can be accompanied by malnutrition or inadequate protein consumption. Sarcopenia is also a major contributor to frailty, the risk of falling, and fall-related bone fractures. Underlying symptoms of frailty include the progressive loss of robust function in multiple tissues and organ systems, and can lead to decreased muscular support of skeletal structure. As such, sarcopenia provides a substantial decrease in quality of life for older adults.

The current primary treatment for sarcopenia is exercise, specifically resistance training or strength training. It is believed that these activities increase muscle strength and endurance. It has also been suggested that hormone replacement, such as testosterone or growth hormone supplementation, may be effective in the treatment of sarcopenia. However, there is no evidence that these treatments result in great improvements in muscle function or reversal of symptoms of sarcopenia. While recent studies have shown that the tryptophan metabolite kynurenine increases in circulation in age in mice and may play a role in sarcopenia (El Refaey, M. et al. J Bone Mineral Research 32:2182-93 (2017)), the cellular and molecular mechanisms involved in age-related muscle wasting are not well-understood. Accordingly, in view of the substantially increasing age of the population, there remains a need for an effective treatment to prevent and/or reduce the onset and advancement of age-related muscle wasting in older adults.

Therefore it is an object of the invention to provide compositions and methods for preventing or treating age-related muscle loss.

### SUMMARY OF THE INVENTION

Compositions of IDO inhibitors are provided that are useful for, for example, treating or preventing muscle loss, increasing or maintaining muscle mass, muscle strength and/or muscle function, treating or preventing sarcopenia, improving muscle functionality, and inhibiting or reducing kynurenine production in the blood.

One embodiment provides a method for preventing or treating muscle loss in a subject in need thereof, including administering to the subject an effective amount of at least one indoleamine 2,3-dioxygenase (“IDO”) inhibitor to stop or reverse the progression of muscle loss in the subject. In some embodiments, the at least one IDO inhibitor may be 1-methyl-D-tryptophan. In other embodiments, the subject has or is susceptible of developing sarcopenia. In still another embodiment, the at least one IDO inhibitor is administered in an effective amount of about 200 to about 2500 mg/kg body weight.

Another embodiment provides a method for preventing or treating sarcopenia in a subject in need thereof, including administering to the subject a therapeutically effective amount of a pharmaceutical composition including an effective amount of at least one IDO inhibitor and a pharmaceutically acceptable excipient to treat or prevent sarcopenia. In one embodiment, the at least one IDO inhibitor is 1-methyl-D-tryptophan. In another embodiment, the subject has or is susceptible of developing sarcopenia. In other embodiments, the pharmaceutical composition is formulated for oral delivery. In still other embodiments, the pharmaceutical composition is formulated as an extended release formulation. In yet another embodiment, the pharmaceutical composition is administered to the subject in a therapeutically effective amount of about 200 to about 2500 mg/kg body weight.

Still another embodiment provides a method for maintaining or increasing muscle mass and/or muscle strength in a subject in need thereof, including administering to the subject an effective amount of at least one IDO inhibitor to increase muscle mass and/or muscle strength in the subject. In one embodiment, the subject has or is susceptible of developing sarcopenia. In another embodiment, the at least one IDO inhibitor is 1-methyl-D-tryptophan, 1-methyl-L-tryptophan, methylthiohydantoin-dl-tryptophan, or any combination thereof. For example, the at least one IDO inhibitor may be 1-methyl-D-tryptophan. In still another embodiment, the muscle mass and/or muscle strength of the subject is increased by at least 10 percent when compared to levels of muscle mass and/or muscle strength prior to administration.

### BRIEF DESCRIPTION OF THE DRAWINGS

Further features and advantages of the invention can be ascertained from the following detailed description that is provided in connection with the drawings described below:

FIG. 1A is a bar graph showing that kynurenine treatment increases reactive oxygen species in C2C12 myoblasts. The X-axis represents treatment group (vehicle (VEH), 1 μm kynurenine (1 μm KYN), and 10 μm kynurenine (10 μm KYN) and the Y-axis represent H₂O₂ concentration/protein concentration. FIG. 1B is a bar graph showing that kynurenine treatment increases reactive oxygen species in human myoblasts. The X-axis represents treatment group (vehicle (VEH) and 10 μm kynurenine (Kyn). FIG. 1C is a bar graph showing changes in quadriceps mass compared to total body mass for young and old mice treated with vehicle (VEH) or kynurenine (KYN) for 4 weeks. The X-axis represents treatment group and the Y-axis represents quadriceps mass per total body weight (g). FIG. 1D is a bar graph showing changes in muscle fiber size in young and old mice treated with vehicle (VEH) or kynurenine (Kyn) for 4 weeks. The X-axis represents treatment group and the Y-axis represents fiber size (μm). FIG. 1E shows lipid peroxidation in young and old mice treated with vehicle (VEH) or kynurenine (Kyn) as measured by 4HNE staining. The X-axis represents treatment group and the Y-axis represents percent 4NHE staining. FIG. 1F is a bar graph showing gene expression in quadriceps muscles from young mice treated with vehicle (VEH) or kynurenine (Kyn). The X-axis represents treatment group and the specific gene (Myh1, Myh2, Murfl, or MAFbx) and the Y-axis represents fold change. FIG. 1G is a bar graph showing muscle strength in mice before and after treatment with kynurenine. The X-axis represents treatment and the Y-axis represents muscle strength (mN-M/body weight).

FIG. 2A is a bar graph showing changes in quadriceps mass compared to total body mass for mice treated with vehicle (Veh) and low or high doses of the IDO inhibitor 1-methyl-D-tryptophan (1-MT). The X-axis represents treatment group and the Y-axis represents quadriceps weight/total body weight. FIG. 2B is a bar graph showing average muscle fiber area (μm) for mice treated with vehicle (Veh) and low or high doses of the IDO inhibitor 1-methyl-D-tryptophan (1-MT). The X-axis represents treatment group and the Y-axis represents average muscle fiber area (μm). FIG. 2C is a bar graph showing H₂O₂ levels in quadriceps from mice treated with vehicle (Veh) or high dose 1-MT. The X-axis represents treatment group and the Y-axis represents H₂O₂/protein content. FIG. 2D is a bar graph showing muscle strength in mice before and after treatment with 1-MT. The X-axis represents treatment group and the Y-axis represents mN-M/body weight.

FIG. 3A is a bar graph showing mitochondrial very long chain acyl-coa dehydrogenase (VLCADm) protein expression in quadriceps from mice treated with kynurenine (Kyn) or 1-MT. The X-axis represents treatment group and the Y-axis represents fold change from controls. FIG. 3B is a Western blot showing expression of VLCADm and β-actin in primary human cells treated with 1 μm or 10 μm kynurenine (KYN) or control. FIG. 3C is a bar graph showing the results of the Western blot in FIG. 3B. The X-axis represents treatment group and the Y-axis represents VLCADm expression divided by β-actin expression.

FIG. 4 is a proteomic analysis showing that levels of myosin 4 increases in aged mice with 1-methyl-D-tryptophan treatment whereas factors associated with muscle oxidative stress decreases with 1-methyl-D-tryptophan treatment;

FIG. 5 is a graph showing that functional enrichment in TOPPGENE of proteins upregulated in aged mouse muscle after 1-methyl-D-tryptophan treatment reveals increased levels of factors associated with muscle protein synthesis; and

FIG. 6 is a graph showing that functional enrichment in TOPPGENE of proteins downregulated in aged mouse muscle after 1-methyl-D-tryptophan treatment reveals decreased levels of factors associated with muscle degradation (ubiquitin ligases) and oxidative stress.

FIG. 7A is a bar graph showing H₂O₂ concentration in protein from quadriceps muscles of mice treated with vehicle, 1 μm kynurenine, 10 μm kynurenine, CH-223191, 1 μm kynurenine+10 μm CH-223191, or 10 μm kynurenine+10 μm CH-223191. The X-axis represents treatment group and the Y-axis represents H₂O₂ concentration/protein content. FIG. 7B is a bar graph showing quadriceps weight per total body weight in Ahr KO mice treated with vehicle or kynurenine. The X-axis represents treatment group and the Y-axis represents quadriceps weight/total body weight.

### DETAILED DESCRIPTION OF THE INVENTION

### I. Definitions

As used herein, the singular forms “a,” “an,” and “the” include plural referents unless the context clearly dictates otherwise. For example, reference to “the method of treatment” includes reference to equivalent steps and methods known to those skilled in the art, and so forth.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments, the values may range in value either above or below the stated value in a range of approx. +/−5%; in other embodiments, the values may range in value either above or below the stated value in a range of approx. +/−2%; in other embodiments, the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “pharmaceutically-acceptable carrier” refers to one or more compatible solid or liquid fillers, diluents, or encapsulating substances that does not cause significant irritation to a human or other vertebrate animal and does not abrogate the biological activity and properties of the administered compound.

The term “carrier” or “excipient” refers to an organic or inorganic, natural or synthetic inactive ingredient in a formulation, with which one or more active ingredients are combined. In some embodiments, a carrier or an excipient is an inert substance added to a pharmaceutical composition to further facilitate administration of a compound, and/or does not cause significant irritation to an organism and does not abrogate the biological activity and properties of the administered compound.

The terms “individual,” “subject,” and “patient” are used interchangeably herein, and refer to a mammal, including, but not limited to, humans, rodents, such as mice and rats, and other laboratory animals.

The term “inhibit,” “suppress,” “decrease,” “interfere,” and/or “reduce” (and like terms) generally refers to the act of reducing, either directly or indirectly, a function, activity, or behavior relative to the natural, expected, or average or relative to current conditions. It is understood that this is typically in relation to some standard or expected value, in other words it is relative, but that it is not always necessary for the standard or relative value to be referred to.

The term “increase,” “enhance,” “stimulate,” and/or “induce” (and like terms) generally refers to the act of improving or increasing, either directly or indirectly, a function or behavior relative to the natural, expected, or average or relative to current conditions.

The terms “treat,” “treating,” or “treatment” refers to alleviating, reducing, or inhibiting one or more symptoms or physiological aspects of a disease, disorder, syndrome, or condition. “Treatment” as used herein covers any treatment of a disease in a subject, and includes: (a) preventing the disease or symptom from occurring in a subject which may be predisposed to the disease or symptom, but has not yet been diagnosed as having it; (b) inhibiting the disease symptom, i.e., arresting its development; or (c) relieving the disease symptom, i.e., causing regression of the disease or symptom.

The terms “prevent,” “prevention,” or “prophylaxis” (and like terms) refers to methods in which the risk of developing disease or condition is reduced. Prophylaxis includes reduction in the risk of developing a disease or condition and/or a prevention of worsening of symptoms or progression of a disease or reduction in the risk of worsening of symptoms or progression of a disease or condition.

The term “onset” refers to the beginning of detectable traits or symptoms of a disease or condition.

### II. Compositions

Compositions including an effective amount of at least one indoleamine 2,3-dioxygenase (“IDO”) inhibitor for treating or preventing muscle loss and/or sarcopenia are disclosed. Without being bound by any particular theory, it is believed that kynurenine, a tryptophan metabolite that increases with age, induces skeletal muscle atrophy and increases reactive oxygen species and oxidative stress in skeletal muscle. This, in turn, leads to the age-related muscle wasting condition, sarcopenia. Through the use of the disclosed compositions including at least one IDO inhibitor, the production of kynurenine, which is produced as a metabolite of tryptophan by the IDO enzyme, can be reduced and/or inhibited. In another embodiment, the disclosed compositions including at least one IDO inhibitor may directly degrade kynurenine leading to the treatment and/or prevention of muscle loss.

In one embodiment, the disclosed compositions include one or more IDO inhibitors. The term “IDO inhibitor” refers to an agent capable of inhibiting the activity of indoleamine 2,3-dioxygenase (IDO). The enzyme has 2 isoforms, IDO1 and IDO2, which act as the first step in the metabolic pathway that breaks down the essential amino acid tryptophan to N-formyl-kynurenine. The IDO inhibitor may inhibit IDO1 and/or IDO2. The IDO inhibitor may be a competitive, noncompetitive, or irreversible IDO inhibitor. A “competitive IDO inhibitor” is a compound that reversibly inhibits IDO enzyme activity at the catalytic site (for example, without limitation, 1-methyl-tryptophan); a “noncompetitive IDO inhibitor” is a compound that reversibly inhibits IDO enzyme activity at a non-catalytic site (for example, without limitation, norharman); and an “irreversible IDO inhibitor” is a compound that irreversibly destroys IDO enzyme activity by forming a covalent bond with the enzyme (for example, without limitation, cyclopropyl/aziridinyl tryptophan derivatives).

The disclosed compositions may include any IDO inhibitor(s) that are capable of inhibiting the activity of IDO. Suitable IDO inhibitors contemplated by the present invention include, but are not limited to, 1-methyl-D-tryptophan, 1-methyl-L-tryptophan, methylthiohydantoin-dl-tryptophan (MTH-Trp), β-(3-benzofuranyl)-DL-alanine, beta-(3-benzo(b)thienyl)-DL-alanine, 6-nitro-L-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-DL-tryptophan, 5-bromoindoxyl diacetate, Naphthoquinone-based, S-allyl-brassinin, S-benzyl-brassinin, 5-Bromo-brassinin, Phenylimidazole-based, 4-phenylimidazole, Exiguamine A, NSC401366, and NLG802. In one embodiment, the disclosed compositions include at least one IDO inhibitor selected from 1-methyl-D-tryptophan, 1-methyl-L-tryptophan, methylthiohydantoin-dl-tryptophan, or any combination thereof. In another embodiment, the disclosed compositions include the IDO inhibitor, 1-methyl-D-tryptophan. In another embodiment, the disclosed compositions include the IDO inhibitor, 1-methyl-L-tryptophan.

A. Pharmaceutical Compositions

One embodiment provides pharmaceutical compositions including an effective amount of at least one IDO inhibitor, for example, 1-methyl-D-tryptophan. In general, pharmaceutical compositions are provided including effective amounts of at least one IDO inhibitor, and optionally pharmaceutically acceptable diluents, preservatives, solubilizers, emulsifiers, adjuvants, excipients, and/or carriers. The pharmaceutical compositions can be formulated for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some in vivo approaches, the compositions disclosed herein are administered to a subject in a therapeutically effective amount. As used herein, the term “effective amount” or “therapeutically effective amount” means a dosage sufficient to treat, inhibit, or alleviate one or more symptoms of the disease being treated or to otherwise provide a desired pharmacologic and/or physiologic effect. The precise dosage will vary according to a variety of factors such as subject-dependent variables (for example, age, immune system health, etc.).

In this aspect, the selected dosage depends upon the desired therapeutic effect, on the route of administration, and on the duration of the treatment desired. However, for the disclosed compositions, generally dosage levels of about 200 to about 2500 mg/kg body weight are administered to mammals. In some embodiments, the disclosed compositions may be administered to a subject in a dosage level of about 500 to about 2000 mg/kg body weight. In other embodiments, the disclosed compositions may be administered to a subject in a dosage level of about 750 to about 1500 mg/kg body weight. Generally, for intravenous injection or infusion, the dosage may be lower.

In some embodiments, the compositions disclosed herein are administered in combination with one or more additional active agents, for example, small molecules or mAB. The combination therapies can include administration of the active agents together in the same admixture, or in separate admixtures. Therefore, in some embodiments, the pharmaceutical composition includes two, three, or more active agents. The pharmaceutical compositions can be formulated as a pharmaceutical dosage unit, referred to as a unit dosage form. Such compositions typically include an effective amount of one or more of the disclosed compounds. The different active agents can have the same or different mechanisms of action. In some embodiments, the combination results in an additive effect on the treatment of the disease or disorder. In some embodiments, the combinations result in a more than additive effect on the treatment of the disease or disorder.

In certain embodiments, the disclosed compositions are administered locally, for example, by injection directly into a site to be treated. In other embodiments, the compositions are injected or otherwise administered directly into the vasculature onto vascular tissue at or adjacent to the intended site of treatment. Typically, the local administration causes an increased localized concentration of the composition which is greater than that which can be achieved by systemic administration.

1. Formulations for Parenteral Administration

In some embodiments, the compositions disclosed herein are formulated for parenteral injection, for example, in an aqueous solution. The formulation may also be in the form of a suspension or emulsion. As discussed above, pharmaceutical compositions are provided including effective amounts of one or more IDO inhibitors, and optionally pharmaceutically acceptable diluents, preservatives, solubilizers, emulsifiers, adjuvants, excipients, and/or carriers. Such compositions may optionally include one or more of the following: diluents, sterile water, buffered saline of various buffer content (for example, Tris-HCl, acetate, phosphate), pH and ionic strength, ionic liquids, and HPβCD; and additives such as detergents and solubilizing agents (for example, TWEEN®20 (polysorbate-20), TWEEN®80 (polysorbate-80)), anti-oxidants (for example, ascorbic acid, sodium metabisulfite), and preservatives (for example, Thimersol, benzyl alcohol) and bulking substances (for example, lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Formulations for Oral Administration

In some embodiments, the compositions are formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the disclosed. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979.

The agents can be chemically modified so that oral delivery of the derivative is efficacious. Generally, the chemical modification contemplated is the attachment of at least one moiety to the component molecule itself, where the moiety permits uptake into the blood stream from the stomach or intestine, or uptake directly into the intestinal mucosa. Also desired is the increase in overall stability of the component or components and increase in circulation time in the body. PEGylation is an exemplary chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J Appl. Biochem. 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. The compositions can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects.

For oral formulations, the location of release may be the stomach, the small intestine (the duodenum, the jejunem, or the ileum), or the large intestine. In some embodiments, the release will avoid the deleterious effects of the stomach environment, either by protection of the agent (or derivative) or by release of the agent (or derivative) beyond the stomach environment, such as in the intestine. To ensure full gastric resistance, a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films.

3. Formulations for Topical Administration

The disclosed compositions can be applied topically. For example, the disclosed compositions can be formulated for application to the lungs, nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator.

Transdermal formulations may also be prepared. These will typically be ointments, lotions, sprays, or patches, all of which can be prepared using standard technology. Transdermal formulations may require the inclusion of penetration enhancers.

4. Controlled Delivery Polymeric Matrices

The compositions disclosed herein can also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where the agent is dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of the disclosed compositions, although in some embodiments biodegradable matrices are preferred. These may be natural or synthetic polymers, although synthetic polymers are preferred in some embodiments due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### III. Methods of Use

The disclosed compositions can be used, for example, to treat or prevent muscle loss, to increase or maintain muscle mass, muscle strength and/or muscle function, to treat or prevent sarcopenia, to improve muscle functionality, and to inhibit or reduce kynurenine production in the blood.

In some embodiments, the effect of the composition on a subject is compared to a control. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or an average determined from measuring the symptom, pharmacologic, or physiologic indicator in one or more subjects that do not have the disease or condition to be treated (for example, healthy subjects). In some embodiments, the effect of the treatment is compared to a conventional treatment that is known in the art. For example, if the disease to be treated is cancer, the conventional treatment could be a chemotherapeutic agent.

A. Methods of Treating or Preventing Muscle Loss

As discussed above, without being by any particular theory, it is believed that inhibition of kynurenine production and/or degradation of kynurenine through the use of the disclosed compositions can provide a therapeutic strategy for the prevention and treatment of muscle loss, for example, age-related muscle loss. Methods of using the disclosed compositions to treat or prevent muscle loss in a subject are provided. Muscle loss includes the progressive loss of muscle mass and/or the progressive weakening and degeneration of muscles, including the skeletal or voluntary muscles (which control movement), cardiac muscles (which control the heart (cardiomyopathies)), and smooth muscles. Methods typically include administering a subject in need thereof an effective amount of at least one IDO inhibitor to slow the progression of, stop the progression of, and/or reverse the progression of muscle loss.

For example, the methods of the present invention may increase or maintain muscle mass, muscle strength, and/or muscle function in a subject. In this aspect, administration of the disclosed compositions may lead to an increase in muscle mass, muscle strength, and/or muscle function by 5%, 10%, 20%, 30%, 40%, 50%, 60%, 70%, 80%, 90%, or more than 90%. Muscle mass can be measured using any method known in the art. For example, muscle mass can be quantified by using an imaging technique, such as computed tomography (CT scan) and magnetic resonance imaging (MM). Muscle strength may also be measured using any technique known in the art, for example, by isometric muscle strength testing methods, isometric manual muscle testing, and comparison of force and displacement measurements.

In some embodiments, the disclosed compositions may be used to treat or prevent diseases or conditions associated with muscle loss. Representative conditions and diseases associated with muscle loss and muscle atrophy that can be inhibited or treated by the disclosed compositions include, but are not limited to, sarcopenia, frailty, amyotrophic lateral sclerosis (ALS), dermatomyositis, Guillain-Barré syndrome, multiple sclerosis, muscular dystrophy, neuropathy, osteoarthritis, rheumatoid arthritis, polio, polymyositis, and spinal muscular atrophy.

In one embodiment, the present invention provides methods of using the disclosed compositions to treat or prevent age-related muscle loss. In this aspect, the present invention provides methods of using the disclosed compositions to treat or prevent sarcopenia in a subject. Methods typically include administering a subject in need thereof an effective amount of at least one IDO inhibitor to prevent, treat, delay, and/or ameliorate the onset, advancement, severity and/or symptoms of sarcopenia. For instance, the methods of the present invention may treat or prevent one or more of the following symptoms associated with sarcopenia: loss of skeletal muscle mass, muscle weakness, fatigue, disability, and morbidity. In another embodiment, the methods of the present invention may increase the strength of skeletal muscle and reduce the risk of bony fractures in subjects with sarcopenia. In still another embodiment, the disclosed methods and compositions may improve exercise ability, increase lean muscle mass, improve survival, and improve quality of life in subjects with sarcopenia.

In this aspect, the treatment is considered to be useful in subjects diagnosed with sarcopenia or in those above the age of 60 at risk of developing sarcopenia; or more generally in the elderly, for example over the age of 65, 70 or 80 years. In this regard, treating sarcopenia also includes delaying the onset of sarcopenia. For example, if a typical male age 60 would begin to see signs of sarcopenia by age 65, treatment could delay the onset by 1, 2, 3, 4, 5, 6, 7, 8, 9, 10, or more years. Thus, according to the methods of the present invention, treating sarcopenia includes treating subjects who have not yet been diagnosed with sarcopenia, but who would be vulnerable or expected to be vulnerable to developing sarcopenia in the future.

In another embodiment, the methods of the present invention may be considered useful for treating subjects who do not yet have muscle wasting, for example, subjects under the age of 65, 60, 55, 50, 45, 40, 35, 30, or 25 who do not have muscle wasting. In still other embodiments, the methods of the present invention may be considered useful for treating subjects who do not have cancer. For example, methods may include administering a subject in need thereof and who does not have cancer an effective amount of at least one IDO inhibitor to prevent, treat, delay, and/or ameliorate the onset, advancement, severity and/or symptoms of sarcopenia.

In other embodiments, the present invention provides methods of using the disclosed compositions to improve muscle functionality. Improvement of muscle functionality encompasses the enhancement of the physical performance of muscles, for example, the enhancement of the physical endurance and fatigue resistance. In this aspect, methods typically include administering a subject in need thereof an effective amount of the disclosed compositions. Administration of the disclosed compositions may lead to an improvement in muscle functionality by as much as 10%, 20%, 30%, 40%, 50%, 60%, 70%, 80%, 90%, or more than 90%.

In still other embodiments, the present invention provides methods of using the disclosed compositions to inhibit or reduce kynurenine production in the blood. The disclosed compositions are administered to a subject in an effective amount to reduce the levels or quantity of kynurenine. In some embodiments, the disclosed compositions lead to direct and/or indirect reduction of kynurenine production by 5%, 10%, 20%, 30%, 40%, 50%, 60%, 70%, 80%, 90%, or more than 90%.

### IV. Co-Therapies

In one embodiment, the disclosed compositions can be administered to a subject in need thereof in combination with: an antimicrobial such as an antibiotic, or an antifungal, or an antiviral, or an antiparasitic, or an essential oil, or a combination thereof.

The disclosed compositions can be administered to a subject in need thereof in combination or alternation with other therapies and therapeutic agents. In some embodiments, the disclosed compositions and the additional therapeutic agent are administered separately, but simultaneously, or in alternation. The disclosed compositions and the additional therapeutic agent can also be administered as part of the same composition. In other embodiments, the disclosed compositions and the second therapeutic agent are administered separately and at different times, but as part of the same treatment regime.

1. Treatment Regimes

The subject can be administered a first therapeutic agent 1, 2, 3, 4, 5, 6, or more hours, or 1, 2, 3, 4, 5, 6, 7, or more days before administration of a second therapeutic agent. In some embodiments, the subject can be administered one or more doses of the first agent every 1, 2, 3, 4, 5, 6 7, 14, 21, 28, 35, or 48 days prior to a first administration of second agent. The disclosed compositions can be the first or the second therapeutic agent.

The disclosed compositions and the additional therapeutic agent can be administered as part of a therapeutic regimen. For example, if a first therapeutic agent can be administered to a subject every fourth day, the second therapeutic agent can be administered on the first, second, third, or fourth day, or combinations thereof. The first therapeutic agent or second therapeutic agent may be repeatedly administered throughout the entire treatment regimen.

Exemplary molecules include, but are not limited to, cytokines, chemotherapeutic agents, radionuclides, other immunotherapeutics, enzymes, antibiotics, antivirals (especially protease inhibitors alone or in combination with nucleosides for treatment of HIV or Hepatitis B or C), anti-parasites (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, ligands that bind to Toll-Like Receptors (including but not limited to CpG oligonucleotides) to activate the innate immune system, molecules that mobilize and optimize the adaptive immune system, other molecules that activate or up-regulate the action of cytotoxic T lymphocytes, natural killer cells and helper T-cells, and other molecules that deactivate or down-regulate suppressor or regulatory T-cells.

The additional therapeutic agents are selected based on the condition, disorder or disease to be treated. For example, the disclosed compositions can be co-administered with one or more additional agents that function to enhance or promote an immune response or reduce or inhibit an immune response.

2. Antimicrobials

One embodiment provides the disclosed compositions and an antimicrobial agent and methods of their use. For example, the disclosed compositions can be administered to the subject in combination with an antimicrobial such as an antibiotic, an antifungal, an antiviral, an antiparasitics, or essential oil.

In some embodiments, the subject is administered the disclosed compositions and/or the antimicrobial at time of admission to the hospital to prevent further bacterial, fungal, or viral complications. The antibiotic can target pathogens.

3. Immunomodulators

a. PD-1 Antagonists

In some embodiments, the disclosed compositions are co-administered with a PD-1 antagonist. Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, which are specifically incorporated by reference herein in their entities, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc. Natl. Acad. Sci. U. S. A, 105:10275-10276 (2008)). Therefore, proteins, antibodies or small molecules that prevent co-ligation of PD-1 and TCR on the T cell membrane are also useful PD-1 antagonists.

In some embodiments, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor. Other PD-1 antagonists contemplated by the methods of this invention include antibodies that bind to PD-1 or ligands of PD-1, and other antibodies.

Suitable anti-PD-1 antibodies include, but are not limited to, those described in the following U.S. Pat. Nos: 7,332,582, 7,488,802, 7,521,051, 7,524,498, 7,563,869, 7,981,416, 8,088,905, 8,287,856, 8,580,247, 8,728,474, 8,779,105, 9,067,999, 9,073,994, 9,084,776, 9,205,148, 9,358,289, 9,387,247, 9,492539, and 9,492,540, all of which are incorporated by reference in their entireties.

See also Berger et al., Clin. Cancer Res., 14:30443051 (2008).

Exemplary anti-B7-H1 (also referred to as anti-PD-L1) antibodies include, but are not limited to, those described in the following U.S. Pat. Nos: 8,383,796, 9,102,725, 9,273,135, 9,393,301, and 9,580,507 all of which are specifically incorporated by reference herein in their entirety.

For anti-B7-DC (also referred to as anti-PD-L2) antibodies see U.S. Pat. Nos. 7,411,051, 7,052,694, 7,390,888, 8,188,238, and 9,255,147 all of which are specifically incorporated by reference herein in their entirety.

Other exemplary PD-1 receptor antagonists include, but are not limited to B7-DC polypeptides, including homologs and variants of these, as well as active fragments of any of the foregoing, and fusion proteins that incorporate any of these. In some embodiments, the fusion protein includes the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, for example from mouse or primate, such as a human, wherein the fragment binds to and blocks PD-1 but does not result in inhibitory signal transduction through PD-1. The fragments can also be part of a fusion protein, for example, an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al., Immunity, Vol. 27, pp. 111-122, (2007)). Such fragments also include the soluble ECD portion of the PD-1 protein that includes mutations, such as the A99L mutation, that increases binding to the natural ligands (Molnar et al., PNAS, 105:10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al., J. Clin. Invest. 119(8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

b. CTLA4 antagonists

Other molecules useful in mediating the effects of T cells in an immune response are also contemplated as additional therapeutic agents. In some embodiments, the molecule is an antagonist of CTLA4, for example an antagonistic anti-CTLA4 antibody. An example of an anti-CTLA4 antibody contemplated for use in the methods of the invention includes an antibody as described in PCT/US2006/043690 (Fischkoff et al., WO/2007/056539).

Dosages for anti-PD-1, anti-B7-H1, and anti-CTLA4 antibody, are known in the art and can be in the range of, for example, 0.1 to 100 mg/kg, or with shorter ranges of 1 to 50 mg/kg, or 10 to 20 mg/kg. An appropriate dose for a human subject can be between 5 and 15 mg/kg, with 10 mg/kg of antibody (for example, human anti-PD-1 antibody) being a specific embodiment.

Specific examples of an anti-CTLA4 antibody useful in the methods of the invention are Ipilimumab, a human anti-CTLA4 antibody, administered at a dose of, for example, about 10 mg/kg, and Tremelimumab a human anti-CTLA4 antibody, administered at a dose of, for example, about 15 mg/kg. See also Sammartino, et al., Clinical Kidney Journal, 3(2):135-137 (2010), published online December 2009.

In other embodiments, the antagonist is a small molecule. A series of small organic compounds have been shown to bind to the B7-1 ligand to prevent binding to CTLA4 (see Erbe et al., J. Biol. Chem., 277:7363-7368 (2002). Such small organics could be administered alone or together with an anti-CTLA4 antibody to reduce inhibitory signal transduction of T cells.

c. Potentiating Agents

In some embodiments, the optional therapeutic agents include a potentiating agent. The potentiating agent acts to increase efficacy of the immune response up-regulator, possibly by more than one mechanism, although the precise mechanism of action is not essential to the broad practice of the present invention.

In some embodiments, the potentiating agent is cyclophosphamide. Cyclophosphamide (CTX, Cytoxan®, or Neosar®) is an oxazahosphorine drug and analogs include ifosfamide (IFO, Ifex), perfosfamide, trophosphamide (trofosfamide; Ixoten), and pharmaceutically acceptable salts, solvates, prodrugs and metabolites thereof (US patent application 20070202077 which is incorporated in its entirety). Ifosfamide (MITOXANA®) is a structural analog of cyclophosphamide and its mechanism of action is considered to be identical or substantially similar to that of cyclophosphamide. Perfosfamide (4-hydroperoxycyclophosphamide) and trophosphamide are also alkylating agents, which are structurally related to cyclophosphamide. For example, perfosfamide alkylates DNA, thereby inhibiting DNA replication and RNA and protein synthesis. New oxazaphosphorines derivatives have been designed and evaluated with an attempt to improve the selectivity and response with reduced host toxicity (Liang J, Huang M, Duan W, Yu X Q, Zhou S. Design of new oxazaphosphorine anticancer drugs. Curr Pharm Des. 2007;13(9):963-78. Review). These include mafosfamide (NSC 345842), glufosfamide (D19575, beta-D-glucosylisophosphoramide mustard), S-(−)-bromofosfamide (CBM-11), NSC 612567 (aldophosphamide perhydrothiazine) and NSC 613060 (aldophosphamide thiazolidine). Mafosfamide is an oxazaphosphorine analog that is a chemically stable 4-thioethane sulfonic acid salt of 4-hydroxy-CPA. Glufosfamide is IFO derivative in which the isophosphoramide mustard, the alkylating metabolite of IFO, is glycosidically linked to a beta-D-glucose molecule. Additional cyclophosphamide analogs are described in U.S. Pat. No. 5,190,929 entitled “Cyclophosphamide analogs useful as anti-tumor agents” which is incorporated herein by reference in its entirety.

Although CTX itself is nontoxic, some of its metabolites are cytotoxic alkylating agents that induce DNA crosslinking and, at higher doses, strand breaks. Many cells are resistant to CTX because they express high levels of the detoxifying enzyme aldehyde dehydrogenase (ALDH). CTX targets proliferating lymphocytes, as lymphocytes (but not hematopoietic stem cells) express only low levels of ALDH, and cycling cells are most sensitive to DNA alkylation agents.

Low doses of CTX (<200 mg/kg) can have immune stimulatory effects, including stimulation of anti-tumor immune responses in humans and mouse models of cancer (Brode & Cooke Crit Rev. Immunol. 28:109-126 (2008)). These low doses are sub-therapeutic and do not have a direct anti-tumor activity. In contrast, high doses of CTX inhibit the anti-tumor response. Several mechanisms may explain the role of CTX in potentiation of anti-tumor immune response: (a) depletion of CD4+CD25+FoxP3+ Treg (and specifically proliferating Treg, which may be especially suppressive), (b) depletion of B lymphocytes; (c) induction of nitric oxide (NO), resulting in suppression of tumor cell growth; (d) mobilization and expansion of CD11b+Gr-1+MDSC. These primary effects have numerous secondary effects; for example following Treg depletion macrophages produce more IFN-γ and less IL-10. CTX has also been shown to induce type I IFN expression and promote homeostatic proliferation of lymphocytes.

Treg depletion is most often cited as the mechanism by which CTX potentiates the anti-tumor immune response. This conclusion is based in part by the results of adoptive transfer experiments. In the AB1-HA tumor model, CTX treatment at Day 9 gives a 75% cure rate. Transfer of purified Treg at Day 12 almost completely inhibited the CTX response (van der Most et al. Cancer Immunol. Immunother. 58:1219-1228 (2009). A similar result was observed in the HHD2 tumor model: adoptive transfer of CD4+CD25+ Treg after CTX pretreatment eliminated therapeutic response to vaccine (Taieb, J. J. Immunol. 176:2722-2729 (2006)).

Numerous human clinical trials have demonstrated that low dose CTX is a safe, well-tolerated, and effective agent for promoting anti-tumor immune responses (Bas, & Mastrangelo Cancer Immunol. Immunother. 47:1-12 (1998)).

The optimal dose for CTX to potentiate an anti-tumor immune response, is one that lowers overall T cell counts by lowering Treg levels below the normal range but is subtherapeutic (see Machiels et al. Cancer Res. 61:3689-3697 (2001)).

In human clinical trials where CTX has been used as an immunopotentiating agent, a dose of 300 mg/m² has usually been used. For an average male (6 ft, 170 pound (78 kg) with a body surface area of 1.98 m²), 300 mg/m² is 8 mg/kg, or 624 mg of total protein. In mouse models of cancer, efficacy has been seen at doses ranging from 15-150 mg/kg, which relates to 0.45-4.5 mg of total protein in a 30 g mouse (Machiels et al. Cancer Res. 61:3689-3697 (2001), Hengst et al Cancer Res. 41:2163-2167 (1981), Hengst Cancer Res. 40:2135-2141 (1980)).

For larger mammals, such as a primate, such as a human, patient, such mg/m² doses may be used but unit doses administered over a finite time interval may also be used. Such unit doses may be administered on a daily basis for a finite time period, such as up to 3 days, or up to 5 days, or up to 7 days, or up to 10 days, or up to 15 days or up to 20 days or up to 25 days, are all specifically contemplated by the invention. The same regimen may be applied for the other potentiating agents recited herein.

In other embodiments, the potentiating agent is an agent that reduces activity and/or number of regulatory T lymphocytes (T-regs), such as Sunitinib (SUTENT®), anti-TGFβ or Imatinib)(GLEEVAC®. The recited treatment regimen may also include administering an adjuvant.

Useful potentiating agents also include mitosis inhibitors, such as paclitaxol, aromatase inhibitors (e.g. Letrozole) and angiogenesis inhibitors (VEGF inhibitors e.g. Avastin, VEGF-Trap) (see, for example, Li et al., Vascular endothelial growth factor blockade reduces intratumoral regulatory T cells and enhances the efficacy of a GM-CSF-secreting cancer immunotherapy. Clin Cancer Res. 2006 Nov. 15; 12(22):6808-16.), anthracyclines, oxaliplatin, doxorubicin, TLR4 antagonists, and IL-18 antagonists.

4. Supplements

In some embodiments, the disclosed compositions are co-administered with a dietary supplement or a nutraceutical. As used herein, the term “nutraceutical” refers to any substance, agent, or combination of agents, that produces a physiological effect in a mammal, such as a medical or health benefit. For example, the disclosed compositions may be co-administered with one or more of the following supplements: creatine (including its salts (e.g., creatine monohydrate), esters (e.g., creatine ethyl ester), chelates, amides, ethers and derivatives thereof), histidine, Vitamin D, Vitamin C, Vitamin B 1, Vitamin B2, Vitamin B3, Vitamin B5, Vitamin B6, Vitamin B12, Vitamin K, a mineral, such as chromium, iron, magnesium, sodium, potassium, vanadium, an amino acid, such as L-arginine, L-ornithine, L-glutamine, L-tyrosine, L-taurine, L-leucine, L-isoleucine, L-theanine and/or L-valine and derivatives thereof, one or more peptides, such as L-carnitine, camosine, anserine, balenine, homocarnosine, kyotorphin, and/or glutathione and derivatives thereof, a methylxanthine, such as caffeine, aminophylline or theophylline, antioxidants, such as lutein, zeaxanthine, a flavanol, such as a flavanol extracted from tea or chocolate, adenosine triphosphates, and combinations thereof.

### EXAMPLES

### Example 1

### Kynurenine Induces Skeletal Muscle Atrophy and Reactive Oxygen Species

Materials and Methods

This example investigated the relationship between kynurenine, a circulating tryptophan metabolite which increases with age, and markers of muscle oxidative stress. C2C12 myoblasts were treated with kynurenine in a dose-dependent manner (0, 1, 10 μM) and reactive oxygen species (“ROS”) was measured using an Amplex red assay. Human myoblasts were also treated with kynurenine (100 μM) and ROS was measured. Female C57BL/6 mice 6 months of age were treated with kynurenine (10 mg/kg BW) or with saline (vehicle control) for 4 weeks. Muscle mass and fiber size were measured from the quadriceps femoris ex vivo, and ROS was measured from paraffin-embedded muscle sections using immunostaining for 4HNE.

Results

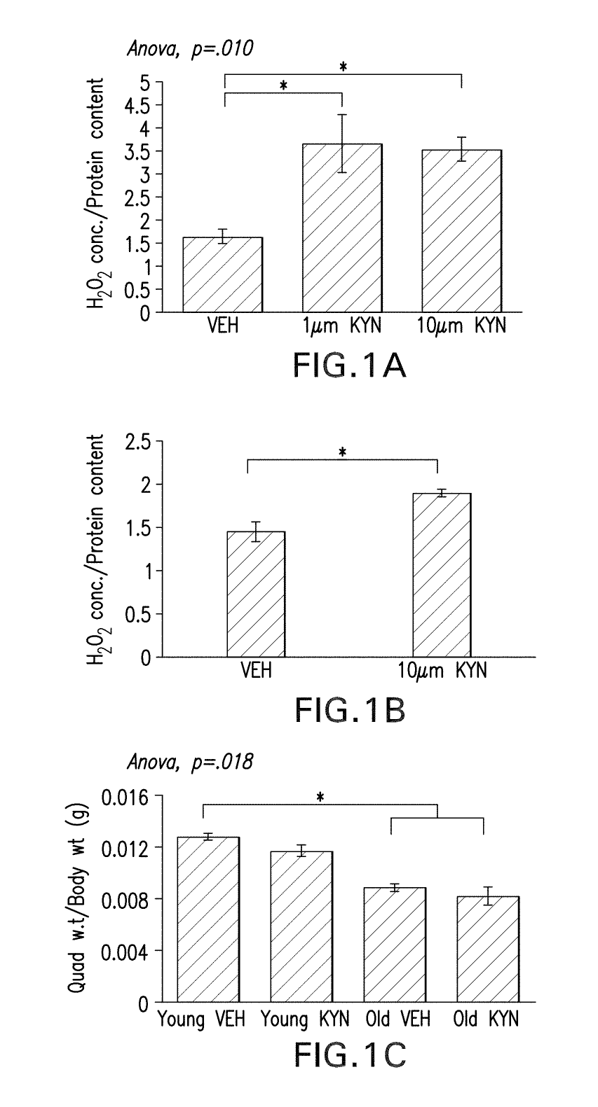

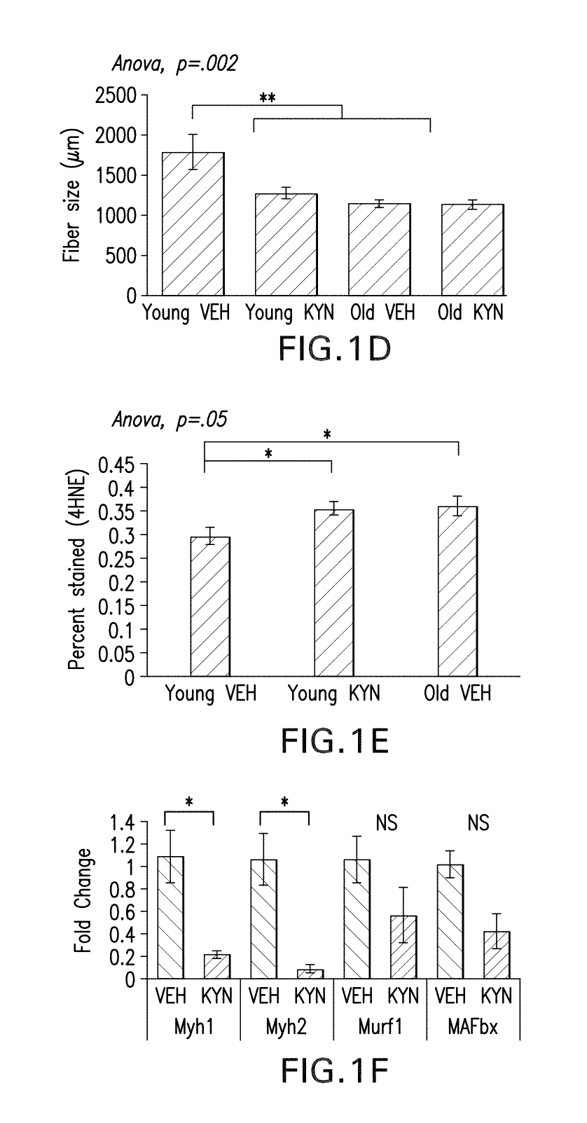

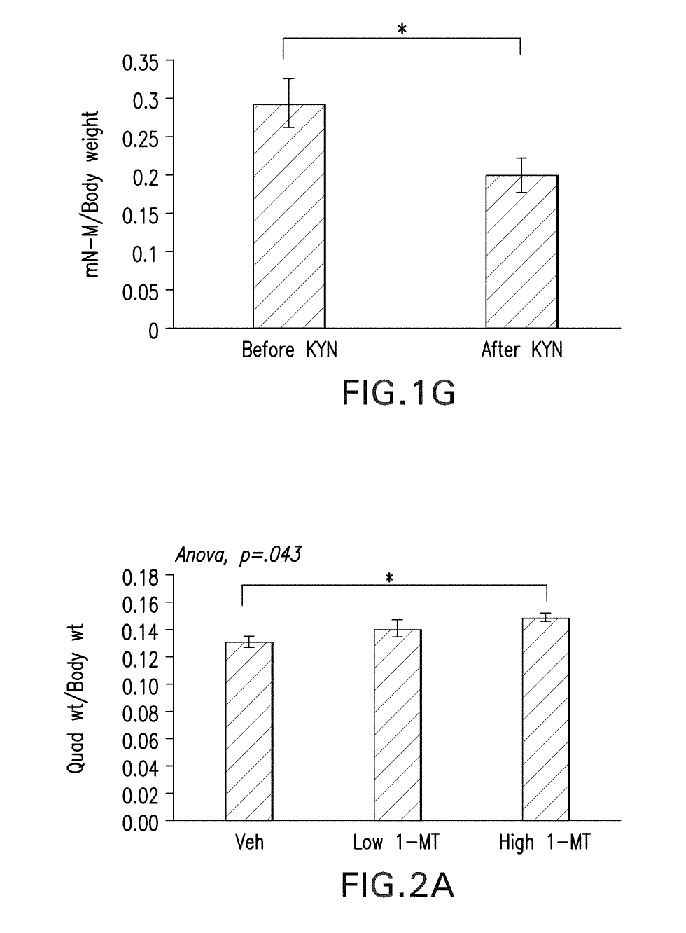

FIGS. 1A and 1B show that treatment with the tryptophan metabolite, kynurenine, increases ROS in muscles in vitro. As shown in FIG. 1A, ROS was increased 2-fold in C2C12 myoblasts treated with kynurenine at both low and high concentrations (P<0.01). FIG. 1B shows that ROS was increased in human myoblasts treated with 100 μM of kynurenine.

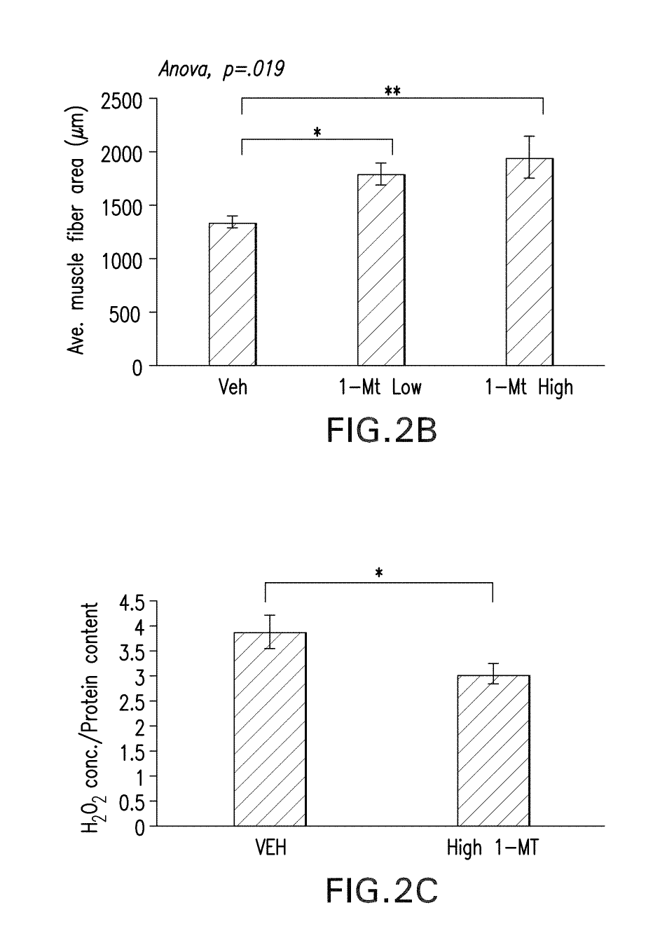

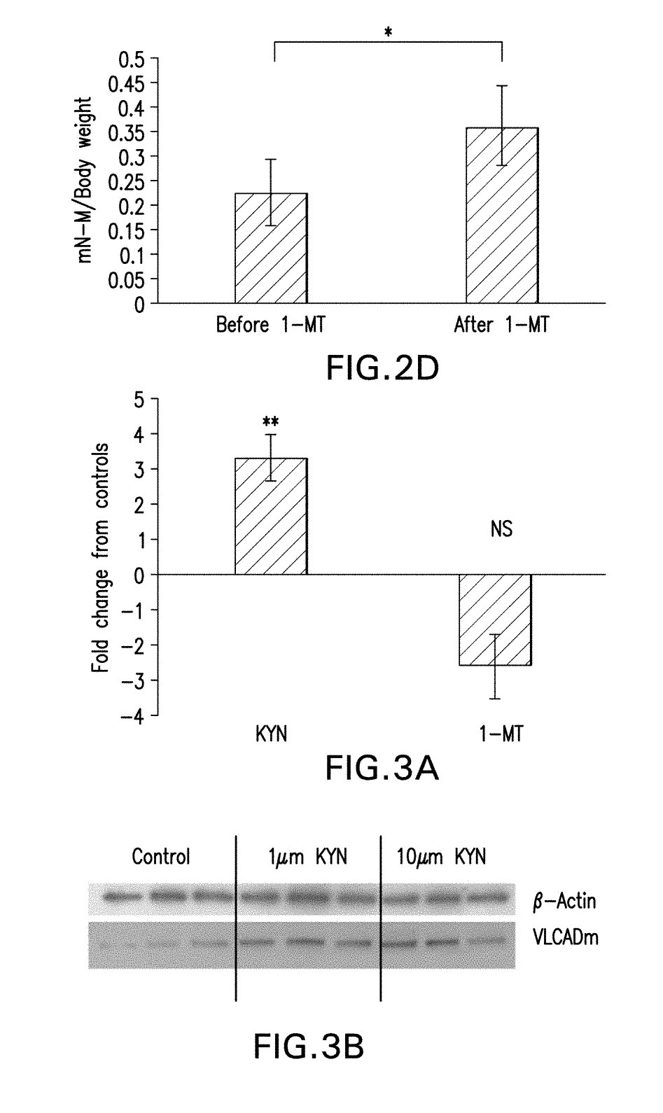

FIGS. 2A and 2B show that treatment with the tryptophan metabolite, kynurenine, decreases muscle mass and muscle fiber size in vivo, respectively. As shown in FIG. 2A, quadriceps weight relative to body weight was reduced (10%) in young mice treated with kynurenine compared to controls. Additionally, as shown in FIG. 2B, muscle fiber size was significantly lower (P<0.01) in young mice treated with kynurenine compared to controls.

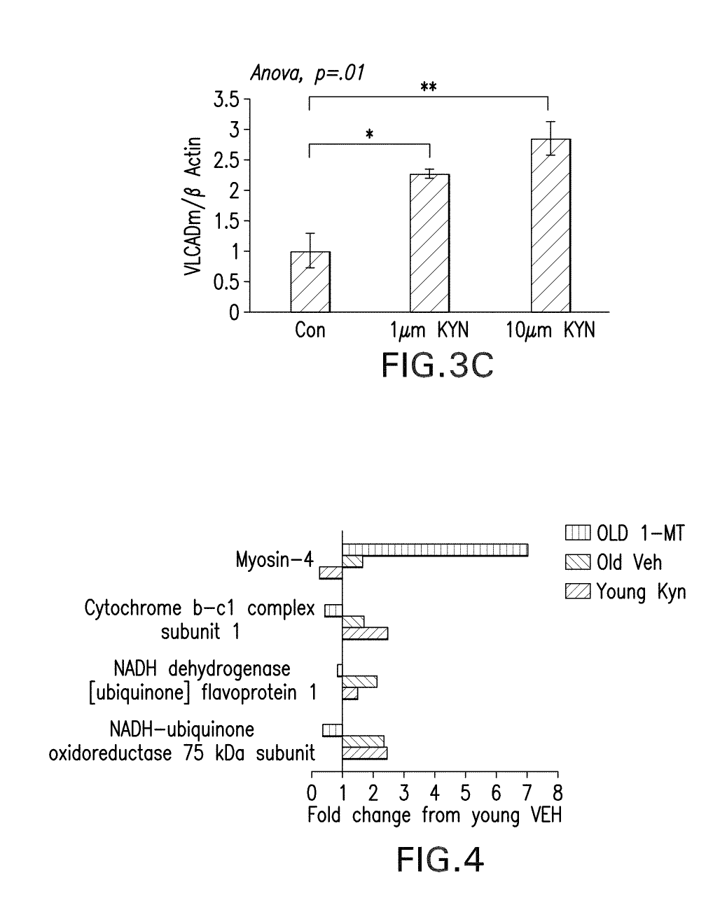

FIG. 3 shows that treatment with the tryptophan metabolite, kynurenine, increases muscle ROS in vivo measured using 4HNE staining. As shown in FIG. 3, ROS was increased by 20% (P<0.05) in young mice treated with kynurenine compared to control based on 4HNE staining.

The above data reveals that the circulating tryptophan metabolite, kynurenine, can induce muscle wasting and increase reactive oxygen species in skeletal muscle. Pharmacological approaches to inhibit kynurenine production may provide a therapeutic strategy for the prevention of sarcopenia.

### Example 2

### IDO Inhibition Blocks Kynurenine Production and Reduces Levels of ROS

Materials and Methods

Female mice 22 months of age were obtained from the National Institute on Aging and treated with saline (vehicle) or 1-methyl-D-tryptophan (1-MT, Sigma, 452483, lot#MKBZ1441V) at a low dose (10 mg/kg BW) or at a high dose (100 mg/kg BW). Mice were treated daily for 4 weeks. Treatments were administered i.p. with an injection volume of 0.2 ml following IACUC approved procedures. Mice were euthanized by CO₂ overdose and muscles harvested for analysis. One quadriceps muscle was snap frozen for proteomics and the other quadriceps muscle fixed in buffered formalin for paraffin embedding and trichrome staining. The tibialis anterior was placed in PBS for amplex red assay.

Results

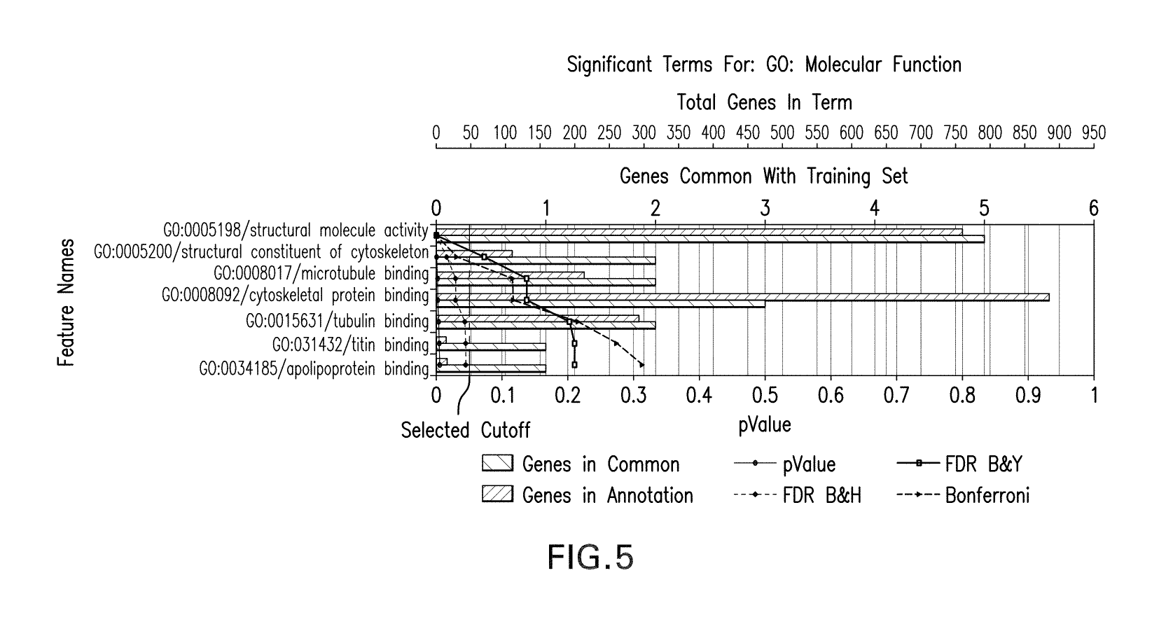

The IDO inhibitor, 1-methyl-D-tryptophan, blocks kynurenine production and reduces levels of ROS in muscle. FIG. 4 shows the levels of ROS in muscle measured using Amplex red assay on mice treated with 1-methyl-D-tryptophan. As shown in FIG. 4, levels of ROS in mice decrease upon treatment with the IDO inhibitor, 1-methyl-D-tryptophan. In addition, proteomic analysis revealed that levels of myosin 4, a key factor for fast, powerful muscle contraction, is increased in aged mice with 1-methyl-D-tryptophan treatment whereas factors associated with muscle oxidative stress are reduced with 1-methyl-D-tryptophan treatment (as shown in FIG. 5). Table 1 below shows the raw proteomic data where aged mice were treated with the 1-methyl-D-tryptophan inhibitor.

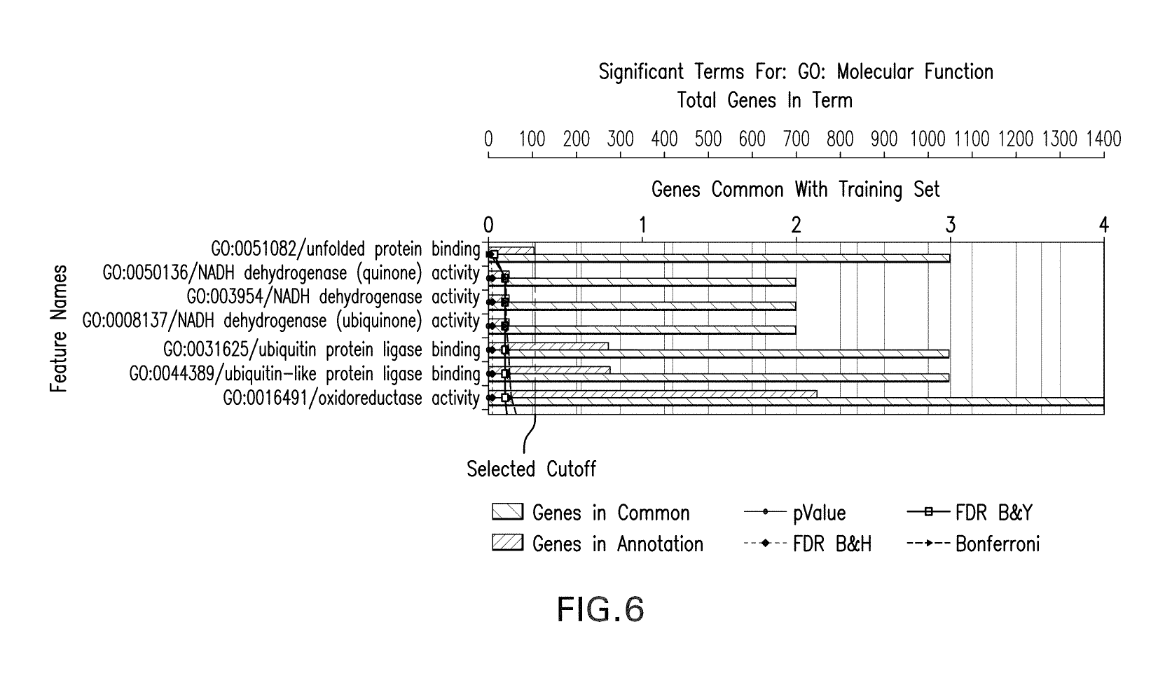

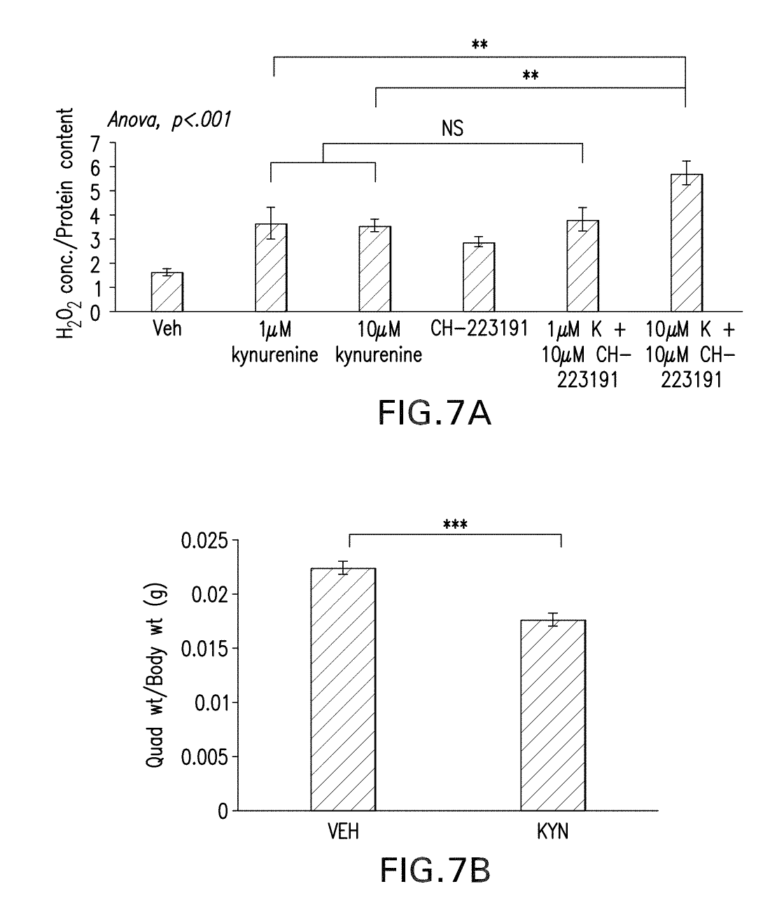

Moreover, as demonstrated in FIG. 6, functional enrichment in TOPPGENE of proteins upregulated in aged mouse muscle after 1-methyl-D-tryptophan treatment reveals increased levels of factors associated with muscle protein synthesis. Functional enrichment in TOPPGENE of proteins downregulated in aged mouse muscle after 1-methyl-D-tryptophan treatment reveals decreased levels of factors associated with muscle degradation (ubiquitin ligases) and oxidative stress, as shown in FIG. 7.

TABLE 1
RAW PROTEOMIC DATA

| | | | unique_pep- | psm_s | unique_pep- | psm_s | 1MT vs |
|---|---|---|---|---|---|---|---|
| | Accession | Description | tides_s 1 | 1 | tides_s 4 | 4 | Control |
| 202 | P68368 | Tubulin alpha-4A chain OS = Mus musculus | 2 | 3 | 3 | 42 | 14 |
| | | GN = Tuba4a PE = 1 SV = 1 - [TBA4A_MOUSE] | | | | | |
| 308 | Q80TF6 | StAR-related lipid transfer protein 9 OS = Mus musculus | 2 | 2 | 4 | 7 | 3.5 |
| | | GN = Stard9 PE = 1 SV = 2 - | | | | | |
| | | [STAR9_MOUSE] | | | | | |
| 412 | Q9CZ30 | Obg-like ATPase 1 OS = Mus musculus GN = Ola1 | 3 | 4 | 5 | 13 | 3.25 |
| | | PE = 1 SV = 1 - [OLA1_MOUSE] | | | | | |
| 80 | P10637 | Microtubule-associated protein tau OS = Mus musculus | 2 | 2 | 4 | 6 | 3 |
| | | GN = Mapt PE = 1 SV = 3 - | | | | | |
| | | [TAU_MOUSE] | | | | | |
| 129 | P27546 | Microtubule-associated protein 4 OS = Mus musculus | 2 | 2 | 4 | 6 | 3 |
| | | GN = Map4 PE = 1 SV = 3 - | | | | | |
| | | [MAP4_MOUSE] | | | | | |
| 452 | Q9ESD7 | Dysferlin OS = Mus musculus GN = Dysf PE = 1 | 2 | 2 | 3 | 6 | 3 |
| | | SV = 3 - [DYSF_MOUSE] | | | | | |
| 494 | Q9Z2U0 | Proteasome subunit alpha type-7 OS = Mus musculus | 2 | 2 | 2 | 6 | 3 |
| | | GN = Psma7 PE = 1 SV = 1 - | | | | | |
| | | [PSA7_MOUSE] | | | | | |
| 343 | Q8CHS7 | Dehydrogenase/reductase SDR family member | 2 | 3 | 3 | 8 | 2.666666667 |
| | | 7C OS = Mus musculus GN = Dhrs7c PE = 1 SV = 3 - | | | | | |
| | | [DRS7C_MOUSE] | | | | | |
| 66 | P07309 | Transthyretin OS = Mus musculus GN = Ttr PE = 1 | 2 | 4 | 2 | 10 | 2.5 |
| | | SV = 1 - [TTHY_MOUSE] | | | | | |
| 325 | Q8BU85 | Methionine-R-sulfoxide reductase B3, | 2 | 2 | 2 | 5 | 2.5 |
| | | mitochondrial OS = Mus musculus GN = Msrb3 | | | | | |
| | | PE = 1 SV = 2 - [MSRB3_MOUSE] | | | | | |
| 398 | Q99MS7 | EH domain-binding protein 1-like protein 1 | 2 | 2 | 2 | 5 | 2.5 |
| | | OS = Mus musculus GN = Ehbp1l1 PE = 1 SV = 1 - | | | | | |
| | | [EH1L1_MOUSE] | | | | | |
| 241 | Q3MI48 | Junctional sarcoplasmic reticulum protein 1 | 3 | 3 | 4 | 7 | 2.333333333 |
| | | OS = Mus musculus GN = Jsrp1 PE = 1 SV = 2 - | | | | | |
| | | [JSPR1_MOUSE] | | | | | |
| 462 | Q9JK53 | Prolargin OS = Mus musculus GN = Prelp PE = 1 | 2 | 3 | 2 | 7 | 2.333333333 |
| | | SV = 2 - [PRELP_MOUSE] | | | | | |
| 1 | A2AAJ9 | Obscurin OS = Mus musculus GN = Obscn PE = 1 | 9 | 27 | 20 | 55 | 2.037037037 |
| | | SV = 2 - [OBSCN_MOUSE] | | | | | |
| 36 | O88342 | WD repeat-containing protein 1 OS = Mus musculus | 2 | 5 | 5 | 10 | 2 |
| | | GN = Wdr1 PE = 1 SV = 3 - | | | | | |
| | | [WDR1_MOUSE] | | | | | |
| 138 | P29758 | Ornithine aminotransferase, mitochondrial | 2 | 2 | 4 | 4 | 2 |
| | | OS = Mus musculus GN = Oat PE = 1 SV = 1 - | | | | | |
| | | [OAT_MOUSE] | | | | | |
| 174 | P56399 | Ubiquitin carboxyl-terminal hydrolase 5 OS = Mus musculus | 2 | 3 | 3 | 6 | 2 |
| | | GN = Usp5 PE = 1 SV = 1 - | | | | | |
| | | [UBP5_MOUSE] | | | | | |
| 186 | P62702 | 40S ribosomal protein S4, X isoform OS = Mus musculus | 2 | 2 | 3 | 4 | 2 |
| | | GN = Rps4x PE = 1 SV = 2 - | | | | | |
| | | [RS4X_MOUSE] | | | | | |
| 298 | Q70IV5 | Synemin OS = Mus musculus GN = Synm PE = 1 | 4 | 4 | 7 | 8 | 2 |
| | | SV = 2 - [SYNEM_MOUSE] | | | | | |
| 4 | A2ASS6 | Titin OS = Mus musculus GN = Ttn PE = 1 SV = 1 - | 479 | 1615 | 696 | 3008 | 1.8625387 |
| | | [TITIN_MOUSE] | | | | | |
| 207 | P70670 | Nascent polypeptide-associated complex subunit | 25 | 55 | 31 | 102 | 1.854545455 |
| | | alpha, muscle-specific form OS = Mus musculus | | | | | |
| | | GN = Naca PE = 1 SV = 2 - [NACAM_MOUSE] | | | | | |
| 78 | P10605 | Cathepsin B OS = Mus musculus GN = Ctsb PE = 1 | 3 | 6 | 3 | 11 | 1.833333333 |
| | | SV = 2 - [CATB_MOUSE] | | | | | |
| 100 | P16045 | Galectin-1 OS = Mus musculus GN = Lgals1 PE = 1 | 4 | 10 | 5 | 18 | 1.8 |
| | | SV = 3 - [LEG1_MOUSE] | | | | | |
| 238 | Q19LI2 | Alpha-1B-glycoprotein OS = Mus musculus | 5 | 10 | 9 | 18 | 1.8 |
| | | GN = A1bg PE = 1 SV = 1 - [A1BG_MOUSE] | | | | | |
| 270 | Q61316 | Heat shock 70 kDa protein 4 OS = Mus musculus | 6 | 10 | 9 | 18 | 1.8 |
| | | GN = Hspa4 PE = 1 SV = 1 - [HSP74_MOUSE] | | | | | |
| 326 | Q8BVI4 | Dihydropteridine reductase OS = Mus musculus | 3 | 7 | 5 | 12 | 1.714285714 |
| | | GN = Qdpr PE = 1 SV = 2 - [DHPR_MOUSE] | | | | | |
| 397 | Q99MR9 | Protein phosphatase 1 regulatory subunit 3A | 5 | 7 | 8 | 12 | 1.714285714 |
| | | OS = Mus musculus GN = Ppp1r3a PE = 1 SV = 2 - | | | | | |
| | | [PPR3A_MOUSE] | | | | | |
| 140 | P32261 | Antithrombin-III OS = Mus musculus | 2 | 3 | 2 | 5 | 1.666666667 |
| | | GN = Serpinc1 PE = 1 SV = 1 - [ANT3_MOUSE] | | | | | |
| 181 | P61971 | Nuclear transport factor 2 OS = Mus musculus | 3 | 6 | 3 | 10 | 1.666666667 |
| | | GN = Nutf2 PE = 1 SV = 1 - [NTF2_MOUSE] | | | | | |
| 329 | Q8C0M9 | Isoaspartyl peptidase/L-asparaginase OS = Mus musculus | 2 | 6 | 3 | 10 | 1.666666667 |
| | | GN = Asrgl1 PE = 1 SV = 1 - | | | | | |
| | | [ASGL1_MOUSE] | | | | | |
| 400 | Q99PT1 | Rho GDP-dissociation inhibitor 1 OS = Mus musculus | 2 | 3 | 2 | 5 | 1.666666667 |
| | | GN = Arhgdia PE = 1 SV = 3 - | | | | | |
| | | [GDIR1_MOUSE] | | | | | |
| 475 | Q9R059 | Four and a half LIM domains protein 3 OS = Mus musculus | 3 | 3 | 3 | 5 | 1.666666667 |
| | | GN = Fhl3 PE = 1 SV = 2 - | | | | | |
| | | [FHL3_MOUSE] | | | | | |
| 71 | P07934 | Phosphorylase b kinase gamma catalytic chain, | 5 | 11 | 5 | 18 | 1.636363636 |
| | | skeletal muscle/heart isoform OS = Mus musculus | | | | | |
| | | GN = Phkg1 PE = 1 SV = 3 - [PHKG1_MOUSE] | | | | | |
| 461 | Q9JK37 | Myozenin-1 OS = Mus musculus GN = Myoz1 | 2 | 8 | 4 | 13 | 1.625 |
| | | PE = 1 SV = 1 - [MYOZ1_MOUSE] | | | | | |
| 272 | Q61554 | Fibrillin-1 OS = Mus musculus GN = Fbn1 PE = 1 | 8 | 13 | 9 | 21 | 1.615384615 |
| | | SV = 1 - [FBN1_MOUSE] | | | | | |
| 331 | Q8C494 | Proline-rich protein 33 OS = Mus musculus | 4 | 5 | 4 | 8 | 1.6 |
| | | GN = Prr33 PE = 2 SV = 1 - [PRR33_MOUSE] | | | | | |
| 260 | Q60854 | Serpin B6 OS = Mus musculus GN = Serpinb6 | 4 | 7 | 3 | 11 | 1.571428571 |
| | | PE = 1 SV = 1 - [SPB6_MOUSE] | | | | | |
| 448 | Q9DCZ1 | GMP reductase 1 OS = Mus musculus GN = Gmpr | 5 | 14 | 6 | 22 | 1.571428571 |
| | | PE = 1 SV = 1 - [GMPR1_MOUSE] | | | | | |
| 476 | Q9R062 | Glycogenin-1 OS = Mus musculus GN = Gyg1 | 6 | 26 | 10 | 40 | 1.538461538 |
| | | PE = 1 SV = 3 - [GLYG_MOUSE] | | | | | |
| 113 | P20029 | 78 kDa glucose-regulated protein OS = Mus musculus | 7 | 23 | 9 | 35 | 1.52173913 |
| | | GN = Hspa5 PE = 1 SV = 3 - | | | | | |
| | | [GRP78_MOUSE] | | | | | |
| 89 | P13412 | Troponin I, fast skeletal muscle OS = Mus musculus | 4 | 8 | 3 | 12 | 1.5 |
| | | GN = Tnni2 PE = 2 SV = 2 - | | | | | |
| | | [TNNI2_MOUSE] | | | | | |
| 128 | P26883 | Peptidyl-prolyl cis-trans isomerase FKBP1A | 3 | 4 | 2 | 6 | 1.5 |
| | | OS = Mus musculus GN = Fkbp1a PE = 1 SV = 2 - | | | | | |
| | | [FKB1A_MOUSE] | | | | | |
| 231 | Q04857 | Collagen alpha-1(VI) chain OS = Mus musculus | 2 | 2 | 3 | 3 | 1.5 |
| | | GN = Col6a1 PE = 1 SV = 1 - [CO6A1_MOUSE] | | | | | |
| 285 | Q64105 | Sepiapterin reductase OS = Mus musculus | 5 | 6 | 6 | 9 | 1.5 |
| | | GN = Spr PE = 1 SV = 1 - [SPRE_MOUSE] | | | | | |
| 313 | Q8BGQ7 | Alanine--tRNA ligase, cytoplasmic OS = Mus musculus | 2 | 2 | 2 | 3 | 1.5 |
| | | GN = Aars PE = 1 SV = 1 - | | | | | |
| | | [SYAC_MOUSE] | | | | | |
| 399 | Q99PR8 | Heat shock protein beta-2 OS = Mus musculus | 4 | 8 | 4 | 12 | 1.5 |
| | | GN = Hspb2 PE = 1 SV = 2 - [HSPB2_MOUSE] | | | | | |
| 432 | Q9D7X3 | Dual specificity protein phosphatase 3 OS = Mus musculus | 3 | 8 | 4 | 12 | 1.5 |
| | | GN = Dusp3 PE = 1 SV = 1 - | | | | | |
| | | [DUS3_MOUSE] | | | | | |
| 492 | Q9Z1Z2 | Serine-threonine kinase receptor-associated | 2 | 2 | 3 | 3 | 1.5 |
| | | protein OS = Mus musculus GN = Strap PE = 1 | | | | | |
| | | SV = 2 - [STRAP_MOUSE] | | | | | |
| 283 | Q62446 | Peptidyl-prolyl cis-trans isomerase FKBP3 | 3 | 7 | 3 | 10 | 1.428571429 |
| | | OS = Mus musculus GN = Fkbp3 PE = 1 SV = 2 - | | | | | |
| | | [FKBP3_MOUSE] | | | | | |
| 32 | O70209 | PDZ and LIM domain protein 3 OS = Mus musculus | 4 | 17 | 5 | 24 | 1.411764706 |
| | | GN = Pdlim3 PE = 1 SV = 1 - | | | | | |
| | | [PDLI3_MOUSE] | | | | | |
| 454 | Q9ET78 | Junctophilin-2 OS = Mus musculus GN = Jph2 | 6 | 21 | 9 | 29 | 1.380952381 |
| | | PE = 1 SV = 2 - [JPH2_MOUSE] | | | | | |
| 212 | P97384 | Annexin A11 OS = Mus musculus GN = Anxa11 | 6 | 8 | 6 | 11 | 1.375 |
| | | PE = 1 SV = 2 - [ANX11_MOUSE] | | | | | |
| 135 | P28654 | Decorin OS = Mus musculus GN = Dcn PE = 1 | 6 | 25 | 7 | 34 | 1.36 |
| | | SV = 1 - [PGS2_MOUSE] | | | | | |
| 247 | Q3TXS7 | 26S proteasome non-ATPase regulatory subunit 1 | 3 | 3 | 4 | 4 | 1.333333333 |
| | | OS = Mus musculus GN = Psmd1 PE = 1 SV = 1 - | | | | | |
| | | [PSMD1_MOUSE] | | | | | |
| 288 | Q64727 | Vinculin OS = Mus musculus GN = Vcl PE = 1 | 2 | 3 | 2 | 4 | 1.333333333 |
| | | SV = 4 - [VINC_MOUSE] | | | | | |
| 339 | Q8CGC7 | Bifunctional glutamate/proline--tRNA ligase | 8 | 15 | 11 | 20 | 1.333333333 |
| | | OS = Mus musculus GN = Eprs PE = 1 SV = 4 - | | | | | |
| | | [SYEP_MOUSE] | | | | | |
| 463 | Q9JKB3 | Y-box-binding protein 3 OS = Mus musculus | 2 | 6 | 3 | 8 | 1.333333333 |
| | | GN = Ybx3 PE = 1 SV = 2 - [YBOX3_MOUSE] | | | | | |
| 72 | P08228 | Superoxide dismutase [Cu—Zn] OS = Mus musculus | 2 | 11 | 3 | 14 | 1.272727273 |
| | | GN = Sod1 PE = 1 SV = 2 - | | | | | |
| | | [SODC_MOUSE] | | | | | |
| 125 | P26043 | Radixin OS = Mus musculus GN = Rdx PE = 1 SV = 3 - | 4 | 15 | 4 | 19 | 1.266666667 |
| | | [RADI_MOUSE] | | | | | |
| 185 | P62631 | Elongation factor 1-alpha 2 OS = Mus musculus | 12 | 156 | 8 | 196 | 1.256410256 |
| | | GN = Eef1a2 PE = 1 SV = 1 - [EF1A2_MOUSE] | | | | | |
| 152 | P47791 | Glutathione reductase, mitochondrial OS = Mus musculus | 3 | 4 | 4 | 5 | 1.25 |
| | | GN = Gsr PE = 1 SV = 3 - | | | | | |
| | | [GSHR_MOUSE] | | | | | |
| 191 | P63005 | Platelet-activating factor acetylhydrolase IB | 2 | 4 | 4 | 5 | 1.25 |
| | | subunit alpha OS = Mus musculus GN = Pafah1b1 | | | | | |
| | | PE = 1 SV = 2 - [LIS1_MOUSE] | | | | | |
| 358 | Q8VCM7 | Fibrinogen gamma chain OS = Mus musculus | 2 | 4 | 2 | 5 | 1.25 |
| | | GN = Fgg PE = 1 SV = 1 - [FIBG_MOUSE] | | | | | |
| 354 | Q8R1G2 | Carboxymethylenebutenolidase homolog | 5 | 17 | 5 | 21 | 1.235294118 |
| | | OS = Mus musculus GN = Cmbl PE = 1 SV = 1 - | | | | | |
| | | [CMBL_MOUSE] | | | | | |
| 165 | P51885 | Lumican OS = Mus musculus GN = Lum PE = 1 | 5 | 39 | 5 | 48 | 1.230769231 |
| | | SV = 2 - [LUM_MOUSE] | | | | | |
| 251 | Q3UZA1 | CapZ-interacting protein OS = Mus musculus | 2 | 5 | 2 | 6 | 1.2 |
| | | GN = Rcsd1 PE = 1 SV = 1 - [CPZIP_MOUSE] | | | | | |
| 424 | Q9D172 | ES1 protein homolog, mitochondrial OS = Mus musculus | 4 | 10 | 5 | 12 | 1.2 |
| | | GN = D10Jhu81e PE = 1 SV = 1 - | | | | | |
| | | [ES1_MOUSE] | | | | | |
| 395 | Q99LX0 | Protein deglycase DJ-1 OS = Mus musculus | 7 | 67 | 7 | 80 | 1.194029851 |
| | | GN = Park7 PE = 1 SV = 1 - [PARK7_MOUSE] | | | | | |
| 162 | P50396 | Rab GDP dissociation inhibitor alpha OS = Mus musculus | 4 | 16 | 5 | 19 | 1.1875 |
| | | GN = Gdi1 PE = 1 SV = 3 - | | | | | |
| | | [GDIA_MOUSE] | | | | | |
| 67 | P07310 | Creatine kinase M-type OS = Mus musculus | 25 | 2011 | 25 | 2378 | 1.182496271 |
| | | GN = Ckm PE = 1 SV = 1 - [KCRM_MOUSE] | | | | | |
| 105 | P17742 | Peptidyl-prolyl cis-trans isomerase A OS = Mus musculus | 5 | 34 | 5 | 40 | 1.176470588 |
| | | GN = Ppia PE = 1 SV = 2 - | | | | | |
| | | [PPIA_MOUSE] | | | | | |
| 132 | P28474 | Alcohol dehydrogenase class-3 OS = Mus musculus | 7 | 17 | 7 | 20 | 1.176470588 |
| | | GN = Adh5 PE = 1 SV = 3 - | | | | | |
| | | [ADHX_MOUSE] | | | | | |
| 102 | P16858 | Glyceraldehyde-3-phosphate dehydrogenase | 16 | 983 | 15 | 1147 | 1.166836216 |
| | | OS = Mus musculus GN = Gapdh PE = 1 SV = 2 - | | | | | |
| | | [G3P_MOUSE] | | | | | |
| 261 | Q60864 | Stress-induced-phosphoprotein 1 OS = Mus musculus | 3 | 6 | 3 | 7 | 1.166666667 |
| | | GN = Stip1 PE = 1 SV = 1 - | | | | | |
| | | [STIP1_MOUSE] | | | | | |
| 205 | P70402 | Myosin-binding protein H OS = Mus musculus | 10 | 45 | 12 | 52 | 1.155555556 |
| | | GN = Mybph PE = 2 SV = 2 - [MYBPH_MOUSE] | | | | | |
| 82 | P10649 | Glutathione S-transferase Mu 1 OS = Mus musculus | 7 | 33 | 8 | 38 | 1.151515152 |
| | | GN = Gstm1 PE = 1 SV = 2 - | | | | | |
| | | [GSTM1_MOUSE] | | | | | |
| 110 | P18826 | Phosphorylase b kinase regulatory subunit alpha, | 14 | 35 | 16 | 40 | 1.142857143 |
| | | skeletal muscle isoform OS = Mus musculus | | | | | |
| | | GN = Phka1 PE = 1 SV = 3 - [KPB1_MOUSE] | | | | | |
| 455 | Q9ET80 | Junctophilin-1 OS = Mus musculus GN = Jph1 | 3 | 7 | 4 | 8 | 1.142857143 |
| | | PE = 1 SV = 1 - [JPH1_MOUSE] | | | | | |
| 106 | P17751 | Triosephosphate isomerase OS = Mus musculus | 12 | 169 | 13 | 192 | 1.136094675 |
| | | GN = Tpi1 PE = 1 SV = 4 - [TPIS_MOUSE] | | | | | |
| 139 | P31001 | Desmin OS = Mus musculus GN = Des PE = 1 SV = 3 - | 6 | 15 | 6 | 17 | 1.133333333 |
| | | [DESM_MOUSE] | | | | | |
| 478 | Q9R0Y5 | Adenylate kinase isoenzyme 1 OS = Mus musculus | 10 | 300 | 10 | 340 | 1.133333333 |
| | | GN = Ak1 PE = 1 SV = 1 - [KAD1_MOUSE] | | | | | |
| 201 | P68134 | Actin, alpha skeletal muscle OS = Mus musculus | 7 | 95 | 7 | 107 | 1.126315789 |
| | | GN = Acta1 PE = 1 SV = 1 - [ACTS_MOUSE] | | | | | |
| 300 | Q76MZ3 | Serine/threonine-protein phosphatase 2A 65 kDa | 3 | 8 | 3 | 9 | 1.125 |
| | | regulatory subunit A alpha isoform OS = Mus musculus | | | | | |
| | | GN = Ppp2r1a PE = 1 SV = 3 - [2AAA_MOUSE] | | | | | |
| 279 | Q62234 | Myomesin-1 OS = Mus musculus GN = Myom1 | 54 | 433 | 59 | 482 | 1.113163972 |
| | | PE = 1 SV = 2 - [MYOM1_MOUSE] | | | | | |
| 98 | P15626 | Glutathione S-transferase Mu 2 OS = Mus musculus | 3 | 18 | 3 | 20 | 1.111111111 |
| | | GN = Gstm2 PE = 1 SV = 2 - | | | | | |
| | | [GSTM2_MOUSE] | | | | | |
| 137 | P29699 | Alpha-2-HS-glycoprotein OS = Mus musculus | 5 | 9 | 4 | 10 | 1.111111111 |
| | | GN = Ahsg PE = 1 SV = 1 - [FETUA_MOUSE] | | | | | |
| 242 | Q3TJD7 | PDZ and LIM domain protein 7 OS = Mus musculus | 4 | 18 | 4 | 20 | 1.111111111 |
| | | GN = Pdlim7 PE = 1 SV = 1 - | | | | | |
| | | [PDLI7_MOUSE] | | | | | |
| 371 | Q91VI7 | Ribonuclease inhibitor OS = Mus musculus | 7 | 9 | 7 | 10 | 1.111111111 |
| | | GN = Rnh1 PE = 1 SV = 1 - [RINI_MOUSE] | | | | | |
| 386 | Q924M7 | Mannose-6-phosphate isomerase OS = Mus musculus | 8 | 18 | 8 | 20 | 1.111111111 |
| | | GN = Mpi PE = 1 SV = 1 - [MPI_MOUSE] | | | | | |
| 255 | Q5SX39 | Myosin-4 OS = Mus musculus GN = Myh4 PE = 2 | 50 | 274 | 39 | 303 | 1.105839416 |
| | | SV = 1 - [MYH4_MOUSE] | | | | | |
| 55 | P04117 | Fatty acid-binding protein, adipocyte OS = Mus musculus | 3 | 20 | 4 | 22 | 1.1 |
| | | GN = Fabp4 PE = 1 SV = 3 - | | | | | |
| | | [FABP4_MOUSE] | | | | | |
| 436 | Q9D8N0 | Elongation factor 1-gamma OS = Mus musculus | 14 | 30 | 11 | 33 | 1.1 |
| | | GN = Eef1g PE = 1 SV = 3 - [EF1G_MOUSE] | | | | | |
| 57 | P05064 | Fructose-bisphosphate aldolase A OS = Mus musculus | 20 | 938 | 21 | 1028 | 1.095948827 |
| | | GN = Aldoa PE = 1 SV = 2 - | | | | | |
| | | [ALDOA_MOUSE] | | | | | |
| 403 | Q9CPU0 | Lactoylglutathione lyase OS = Mus musculus | 6 | 42 | 8 | 46 | 1.095238095 |
| | | GN = Glo1 PE = 1 SV = 3 - [LGUL_MOUSE] | | | | | |
| 177 | P58252 | Elongation factor 2 OS = Mus musculus GN = Eef2 | 23 | 142 | 22 | 155 | 1.091549296 |
| | | PE = 1 SV = 2 - [EF2_MOUSE] | | | | | |
| 69 | P07758 | Alpha-1-antitrypsin 1-1 OS = Mus musculus | 3 | 77 | 3 | 84 | 1.090909091 |
| | | GN = Serpina1a PE = 1 SV = 4 - [A1AT1_MOUSE] | | | | | |
| 75 | P09411 | Phosphoglycerate kinase 1 OS = Mus musculus | 22 | 350 | 23 | 381 | 1.088571429 |
| | | GN = Pgk1 PE = 1 SV = 4 - [PGK1_MOUSE] | | | | | |
| 306 | Q7TSH2 | Phosphorylase b kinase regulatory subunit beta | 16 | 48 | 17 | 52 | 1.083333333 |
| | | OS = Mus musculus GN = Phkb PE = 1 SV = 1 - | | | | | |
| | | [KPBB_MOUSE] | | | | | |
| 120 | P23953 | Carboxylesterase 1C OS = Mus musculus | 7 | 25 | 7 | 27 | 1.08 |
| | | GN = Ces1c PE = 1 SV = 4 - [EST1C_MOUSE] | | | | | |
| 5 | A2AUC9 | Kelch-like protein 41 OS = Mus musculus | 13 | 39 | 12 | 42 | 1.076923077 |
| | | GN = Klhl41 PE = 1 SV = 1 - [KLH41_MOUSE] | | | | | |
| 74 | P09103 | Protein disulfide-isomerase OS = Mus musculus | 7 | 26 | 8 | 28 | 1.076923077 |
| | | GN = P4hb PE = 1 SV = 2 - [PDIA1_MOUSE] | | | | | |
| 376 | Q91YE8 | Synaptopodin-2 OS = Mus musculus GN = Synpo2 | 5 | 13 | 7 | 14 | 1.076923077 |
| | | PE = 1 SV = 2 - [SYNP2_MOUSE] | | | | | |
| 90 | P13707 | Glycerol-3-phosphate dehydrogenase [NAD(+)], | 17 | 138 | 16 | 148 | 1.072463768 |
| | | cytoplasmic OS = Mus musculus GN = Gpd1 PE = 1 | | | | | |
| | | SV = 3 - [GPDA_MOUSE] | | | | | |
| 17 | O08539 | Myc box-dependent-interacting protein 1 | 11 | 47 | 10 | 50 | 1.063829787 |
| | | OS = Mus musculus GN = Bin1 PE = 1 SV = 1 - | | | | | |
| | | [BIN1_MOUSE] | | | | | |
| 147 | P45376 | Aldose reductase OS = Mus musculus GN = Akr1b1 | 7 | 47 | 6 | 50 | 1.063829787 |
| | | PE = 1 SV = 3 - [ALDR_MOUSE] | | | | | |
| 116 | P21550 | Beta-enolase OS = Mus musculus GN = Eno3 PE = 1 | 13 | 811 | 12 | 862 | 1.062885327 |
| | | SV = 3 - [ENOB_MOUSE] | | | | | |
| 368 | Q8VHX6 | Filamin-C OS = Mus musculus GN = Flnc PE = 1 | 28 | 68 | 24 | 72 | 1.058823529 |
| | | SV = 3 - [FLNC_MOUSE] | | | | | |
| 486 | Q9WUZ7 | SH3 domain-binding glutamic acid-rich protein | 6 | 35 | 7 | 37 | 1.057142857 |
| | | OS = Mus musculus GN = Sh3bgr PE = 1 SV = 1 - | | | | | |
| | | [SH3BG_MOUSE] | | | | | |
| 103 | P17182 | Alpha-enolase OS = Mus musculus GN = Eno1 | 10 | 436 | 10 | 453 | 1.038990826 |
| | | PE = 1 SV = 3 - [ENOA_MOUSE] | | | | | |
| 464 | Q9JKS4 | LIM domain-binding protein 3 OS = Mus musculus | 18 | 134 | 17 | 139 | 1.037313433 |
| | | GN = Ldb3 PE = 1 SV = 1 - | | | | | |
| | | [LDB3_MOUSE] | | | | | |
| 117 | P22599 | Alpha-1-antitrypsin 1-2 OS = Mus musculus | 3 | 72 | 3 | 74 | 1.027777778 |
| | | GN = Serpina1b PE = 1 SV = 2 - [A1AT2_MOUSE] | | | | | |
| 203 | P70296 | Phosphatidylethanolamine-binding protein 1 | 8 | 83 | 8 | 85 | 1.024096386 |
| | | OS = Mus musculus GN = Pebp1 PE = 1 SV = 3 - | | | | | |
| | | [PEBP1_MOUSE] | | | | | |
| 192 | P63017 | Heat shock cognate 71 kDa protein OS = Mus musculus | 20 | 190 | 21 | 193 | 1.015789474 |
| | | GN = Hspa8 PE = 1 SV = 1 - | | | | | |
| | | [HSP7C_MOUSE] | | | | | |
| 472 | Q9QYG0 | Protein NDRG2 OS = Mus musculus GN = Ndrg2 | 9 | 66 | 9 | 67 | 1.015151515 |
| | | PE = 1 SV = 1 - [NDRG2_MOUSE] | | | | | |
| 154 | P47857 | ATP-dependent 6-phosphofructokinase, muscle | 30 | 449 | 32 | 455 | 1.013363029 |
| | | type OS = Mus musculus GN = Pfkm PE = 1 SV = 3 - | | | | | |
| | | [PFKAM_MOUSE] | | | | | |
| 58 | P05132 | cAMP-dependent protein kinase catalytic subunit | 4 | 10 | 3 | 10 | 1 |
| | | alpha OS = Mus musculus GN = Prkaca PE = 1 | | | | | |
| | | SV = 3 - [KAPCA_MOUSE] | | | | | |
| 93 | P14231 | Sodium/potassium-transporting ATPase subunit | 2 | 5 | 2 | 5 | 1 |
| | | beta-2 OS = Mus musculus GN = Atp1b2 PE = 1 | | | | | |
| | | SV = 2 - [AT1B2_MOUSE] | | | | | |
| 118 | P23506 | Protein-L-isoaspartate(D-aspartate) O- | 2 | 5 | 3 | 5 | 1 |
| | | methyltransferase OS = Mus musculus GN = Pcmt1 | | | | | |
| | | PE = 1 SV = 3 - [PIMT_MOUSE] | | | | | |
| 148 | P45591 | Cofilin-2 OS = Mus musculus GN = Cfl2 PE = 1 | 4 | 32 | 4 | 32 | 1 |
| | | SV = 1 - [COF2_MOUSE] | | | | | |
| 188 | P62908 | 40S ribosomal protein S3 OS = Mus musculus | 2 | 5 | 3 | 5 | 1 |
| | | GN = Rps3 PE = 1 SV = 1 - [RS3_MOUSE] | | | | | |
| 210 | P82349 | Beta-sarcoglycan OS = Mus musculus GN = Sgcb | 2 | 2 | 2 | 2 | 1 |
| | | PE = 1 SV = 1 - [SGCB_MOUSE] | | | | | |
| 246 | Q3TVI8 | Pre-B-cell leukemia transcription factor- | 4 | 5 | 4 | 5 | 1 |
| | | interacting protein 1 OS = Mus musculus | | | | | |
| | | GN = Pbxip1 PE = 1 SV = 2 - [PBIP1_MOUSE] | | | | | |
| 249 | Q3U0V1 | Far upstream element-binding protein 2 OS = Mus musculus | 2 | 2 | 2 | 2 | 1 |
| | | GN = Khsrp PE = 1 SV = 2 - | | | | | |
| | | [FUBP2_MOUSE] | | | | | |
| 269 | Q61292 | Laminin subunit beta-2 OS = Mus musculus | 2 | 2 | 2 | 2 | 1 |
| | | GN = Lamb2 PE = 1 SV = 2 - [LAMB2_MOUSE] | | | | | |
| 273 | Q61584 | Fragile X mental retardation syndrome-related | 2 | 2 | 2 | 2 | 1 |
| | | protein 1 OS = Mus musculus GN = Fxr1 PE = 1 | | | | | |
| | | SV = 2 - [FXR1_MOUSE] | | | | | |
| 299 | Q70KF4 | Cardiomyopathy-associated protein 5 OS = Mus musculus | 8 | 11 | 7 | 11 | 1 |
| | | GN = Cmya5 PE = 1 SV = 2 - | | | | | |
| | | [CMYA5_MOUSE] | | | | | |
| 333 | Q8C7E7 | Starch-binding domain-containing protein 1 | 3 | 5 | 3 | 5 | 1 |
| | | OS = Mus musculus GN = Stbd1 PE = 1 SV = 1 - | | | | | |
| | | [STBD1_MOUSE] | | | | | |
| 342 | Q8CHP8 | Phosphoglycolate phosphatase OS = Mus musculus | 2 | 4 | 4 | 4 | 1 |
| | | GN = Pgp PE = 1 SV = 1 - [PGP_MOUSE] | | | | | |
| 344 | Q8CHT0 | Delta-1-pyrroline-5-carboxylate dehydrogenase, | 3 | 3 | 2 | 3 | 1 |
| | | mitochondrial OS = Mus musculus GN = Aldh4a1 | | | | | |
| | | PE = 1 SV = 3 - [AL4A1_MOUSE] | | | | | |
| 351 | Q8K4Z3 | NAD(P)H-hydrate epimerase OS = Mus musculus | 2 | 4 | 2 | 4 | 1 |
| | | GN = Apoa1bp PE = 1 SV = 1 - [NNRE_MOUSE] | | | | | |
| 355 | Q8R3Z5 | Voltage-dependent L-type calcium channel | 3 | 5 | 3 | 5 | 1 |
| | | subunit beta-1 OS = Mus musculus GN = Cacnb1 | | | | | |
| | | PE = 1 SV = 1 - [CACB1_MOUSE] | | | | | |
| 404 | Q9CPY7 | Cytosol aminopeptidase OS = Mus musculus | 3 | 3 | 2 | 3 | 1 |
| | | GN = Lap3 PE = 1 SV = 3 - [AMPL_MOUSE] | | | | | |
| 413 | Q9CZ44 | NSFL1 cofactor p47 OS = Mus musculus | 4 | 11 | 4 | 11 | 1 |
| | | GN = Nsfl1c PE = 1 SV = 1 - [NSF1C_MOUSE] | | | | | |
| 433 | Q9D819 | Inorganic pyrophosphatase OS = Mus musculus | 2 | 3 | 2 | 3 | 1 |
| | | GN = Ppa1 PE = 1 SV = 1 - [IPYR_MOUSE] | | | | | |
| 434 | Q9D892 | Inosine triphosphate pyrophosphatase OS = Mus musculus | 2 | 4 | 2 | 4 | 1 |
| | | GN = Itpa PE = 1 SV = 2 - | | | | | |
| | | [ITPA_MOUSE] | | | | | |
| 467 | Q9JMH6 | Thioredoxin reductase 1, cytoplasmic OS = Mus musculus | 4 | 6 | 4 | 6 | 1 |
| | | GN = Txnrd1 PE = 1 SV = 3 - | | | | | |
| | | [TRXR1_MOUSE] | | | | | |
| 474 | Q9QZ47 | Troponin T, fast skeletal muscle OS = Mus musculus | 2 | 13 | 3 | 13 | 1 |
| | | GN = Tnnt3 PE = 1 SV = 3 - | | | | | |
| | | [TNNT3_MOUSE] | | | | | |
| 488 | Q9Z0N1 | Eukaryotic translation initiation factor 2 subunit | 2 | 2 | 2 | 2 | 1 |
| | | 3, X-linked OS = Mus musculus GN = Eif2s3x | | | | | |
| | | PE = 1 SV = 2 - [IF2G_MOUSE] | | | | | |
| 497 | Q9Z2Y8 | Proline synthase co-transcribed bacterial homolog | 2 | 2 | 2 | 2 | 1 |
| | | protein OS = Mus musculus GN = Prosc PE = 1 | | | | | |
| | | SV = 1 - [PROSC_MOUSE] | | | | | |
| 304 | Q7TQ48 | Sarcalumenin OS = Mus musculus GN = Srl PE = 1 | 26 | 217 | 25 | 215 | 0.99078341 |
| | | SV = 1 - [SRCA_MOUSE] | | | | | |
| 380 | Q921I1 | Serotransferrin OS = Mus musculus GN = Tf PE = 1 | 28 | 148 | 25 | 146 | 0.986486486 |
| | | SV = 1 - [TRFE_MOUSE] | | | | | |
| 359 | Q8VCR8 | Myosin light chain kinase 2, skeletal/cardiac | 10 | 53 | 12 | 52 | 0.981132075 |
| | | muscle OS = Mus musculus GN = Mylk2 PE = 1 | | | | | |
| | | SV = 2 - [MYLK2_MOUSE] | | | | | |
| 65 | P06801 | NADP-dependent malic enzyme OS = Mus musculus | 12 | 41 | 15 | 40 | 0.975609756 |
| | | GN = Me1 PE = 1 SV = 2 - | | | | | |
| | | [MAOX_MOUSE] | | | | | |
| 99 | P16015 | Carbonic anhydrase 3 OS = Mus musculus | 17 | 403 | 16 | 390 | 0.967741935 |
| | | GN = Ca3 PE = 1 SV = 3 - [CAH3_MOUSE] | | | | | |
| 85 | P11499 | Heat shock protein HSP 90-beta OS = Mus musculus | 14 | 99 | 15 | 95 | 0.95959596 |
| | | GN = Hsp90ab1 PE = 1 SV = 3 - | | | | | |
| | | [HS90B_MOUSE] | | | | | |
| 420 | Q9D0F9 | Phosphoglucomutase-1 OS = Mus musculus | 27 | 358 | 25 | 343 | 0.958100559 |
| | | GN = Pgm1 PE = 1 SV = 4 - [PGM1_MOUSE] | | | | | |
| 16 | O08532 | Voltage-dependent calcium channel subunit | 9 | 22 | 9 | 21 | 0.954545455 |
| | | alpha-2/delta-1 OS = Mus musculus | | | | | |
| | | GN = Cacna2d1 PE = 1 SV = 1 - [CA2D1_MOUSE] | | | | | |
| 193 | P63028 | Translationally-controlled tumor protein OS = Mus musculus | 5 | 18 | 4 | 17 | 0.944444444 |
| | | GN = Tpt1 PE = 1 SV = 1 - | | | | | |
| | | [TCTP_MOUSE] | | | | | |
| 45 | P01837 | Ig kappa chain C region OS = Mus musculus PE = 1 | 3 | 16 | 3 | 15 | 0.9375 |
| | | SV = 1 - [IGKC_MOUSE] | | | | | |
| 199 | P68037 | Ubiquitin-conjugating enzyme E2 L3 OS = Mus musculus | 3 | 15 | 3 | 14 | 0.933333333 |
| | | GN = Ube2l3 PE = 1 SV = 1 - | | | | | |
| | | [UB2L3_MOUSE] | | | | | |
| 485 | Q9WUM5 | Succinyl-CoA ligase [ADP/GDP-forming] | 4 | 15 | 5 | 14 | 0.933333333 |
| | | subunit alpha, mitochondrial OS = Mus musculus | | | | | |
| | | GN = Suclg1 PE = 1 SV = 4 - [SUCA_MOUSE] | | | | | |
| 41 | O89104 | Synaptophysin-like protein 2 OS = Mus musculus | 7 | 43 | 6 | 40 | 0.930232558 |
| | | GN = Sypl2 PE = 1 SV = 1 - [SYPL2_MOUSE] | | | | | |
| 460 | Q9JIF9 | Myotilin OS = Mus musculus GN = Myot PE = 1 | 5 | 14 | 5 | 13 | 0.928571429 |
| | | SV = 1 - [MYOTI_MOUSE] | | | | | |
| 166 | P52480 | Pyruvate kinase PKM OS = Mus musculus | 23 | 751 | 21 | 697 | 0.928095872 |
| | | GN = Pkm PE = 1 SV = 4 - [KPYM_MOUSE] | | | | | |
| 62 | P06151 | L-lactate dehydrogenase A chain OS = Mus musculus | 19 | 528 | 20 | 490 | 0.928030303 |
| | | GN = Ldha PE = 1 SV = 3 - | | | | | |
| | | [LDHA_MOUSE] | | | | | |
| 256 | Q5XKE0 | Myosin-binding protein C, fast-type OS = Mus musculus | 46 | 375 | 44 | 348 | 0.928 |
| | | GN = Mybpc2 PE = 1 SV = 1 - | | | | | |
| | | [MYPC2_MOUSE] | | | | | |
| 274 | Q61598 | Rab GDP dissociation inhibitor beta OS = Mus musculus | 9 | 40 | 8 | 37 | 0.925 |
| | | GN = Gdi2 PE = 1 SV = 1 - | | | | | |
| | | [GDIB_MOUSE] | | | | | |
| 429 | Q9D6R2 | Isocitrate dehydrogenase [NAD] subunit alpha, | 7 | 40 | 7 | 37 | 0.925 |
| | | mitochondrial OS = Mus musculus GN = Idh3a | | | | | |
| | | PE = 1 SV = 1 - [IDH3A_MOUSE] | | | | | |
| 224 | Q01768 | Nucleoside diphosphate kinase B OS = Mus musculus | 6 | 66 | 3 | 61 | 0.924242424 |
| | | GN = Nme2 PE = 1 SV = 1 - | | | | | |
| | | [NDKB_MOUSE] | | | | | |
| 352 | Q8QZT1 | Acetyl-CoA acetyltransferase, mitochondrial | 8 | 26 | 8 | 24 | 0.923076923 |
| | | OS = Mus musculus GN = Acat1 PE = 1 SV = 1 - | | | | | |
| | | [THIL_MOUSE] | | | | | |
| 484 | Q9WUB3 | Glycogen phosphorylase, muscle form OS = Mus musculus | 52 | 981 | 36 | 901 | 0.918450561 |
| | | GN = Pygm PE = 1 SV = 3 - | | | | | |
| | | [PYGM_MOUSE] | | | | | |
| 381 | Q922B1 | O-acetyl-ADP-ribose deacetylase MACROD1 | 3 | 12 | 4 | 11 | 0.916666667 |
| | | OS = Mus musculus GN = Macrod1 PE = 1 SV = 2 - | | | | | |
| | | [MACD1_MOUSE] | | | | | |
| 73 | P08249 | Malate dehydrogenase, mitochondrial OS = Mus musculus | 12 | 153 | 12 | 140 | 0.91503268 |
| | | GN = Mdh2 PE = 1 SV = 3 - | | | | | |
| | | [MDHM_MOUSE] | | | | | |
| 143 | P35700 | Peroxiredoxin-1 OS = Mus musculus GN = Prdx1 | 9 | 58 | 9 | 53 | 0.913793103 |
| | | PE = 1 SV = 1 - [PRDX1_MOUSE] | | | | | |
| 239 | Q1XH17 | Tripartite motif-containing protein 72 OS = Mus musculus | 9 | 42 | 10 | 38 | 0.904761905 |
| | | GN = Trim72 PE = 1 SV = 1 - | | | | | |
| | | [TRI72_MOUSE] | | | | | |
| 357 | Q8R429 | Sarcoplasmic/endoplasmic reticulum calcium | 51 | 1257 | 33 | 1137 | 0.904534606 |
| | | ATPase 1 OS = Mus musculus GN = Atp2a1 PE = 1 | | | | | |
| | | SV = 1 - [AT2A1_MOUSE] | | | | | |
| 225 | Q01853 | Transitional endoplasmic reticulum ATPase | 29 | 125 | 27 | 113 | 0.904 |
| | | OS = Mus musculus GN = Vcp PE = 1 SV = 4 - | | | | | |
| | | [TERA_MOUSE] | | | | | |
| 27 | O09165 | Calsequestrin-1 OS = Mus musculus GN = Casq1 | 14 | 83 | 14 | 75 | 0.903614458 |
| | | PE = 1 SV = 3 - [CASQ1_MOUSE] | | | | | |
| 379 | Q91ZJ5 | UTP--glucose-1-phosphate uridylyltransferase | 10 | 83 | 11 | 75 | 0.903614458 |
| | | OS = Mus musculus GN = Ugp2 PE = 1 SV = 3 - | | | | | |
| | | [UGPA_MOUSE] | | | | | |
| 30 | O54724 | Polymerase I and transcript release factor | 3 | 10 | 2 | 9 | 0.9 |
| | | OS = Mus musculus GN = Ptrf PE = 1 SV = 1 - | | | | | |
| | | [PTRF_MOUSE] | | | | | |
| 196 | P63242 | Eukaryotic translation initiation factor 5A-1 | 6 | 47 | 6 | 42 | 0.893617021 |
| | | OS = Mus musculus GN = Eif5a PE = 1 SV = 2 - | | | | | |
| | | [IF5A1_MOUSE] | | | | | |
| 477 | Q9R0P3 | S-formylglutathione hydrolase OS = Mus musculus | 7 | 28 | 6 | 25 | 0.892857143 |
| | | GN = Esd PE = 1 SV = 1 - | | | | | |
| | | [ESTD_MOUSE] | | | | | |
| 200 | P68040 | Guanine nucleotide-binding protein subunit beta- | 5 | 9 | 5 | 8 | 0.888888889 |
| | | 2-like 1 OS = Mus musculus GN = Gnb2l1 PE = 1 | | | | | |
| | | SV = 3 - [GBLP_MOUSE] | | | | | |
| 68 | P07724 | Serum albumin OS = Mus musculus GN = Alb | 31 | 540 | 29 | 474 | 0.877777778 |
| | | PE = 1 SV = 3 - [ALBU_MOUSE] | | | | | |
| 431 | Q9D7G0 | Ribose-phosphate pyrophosphokinase 1 OS = Mus musculus | 6 | 47 | 7 | 41 | 0.872340426 |
| | | GN = Prps1 PE = 1 SV = 4 - | | | | | |
| | | [PRPS1_MOUSE] | | | | | |
| 204 | P70349 | Histidine triad nucleotide-binding protein 1 | 5 | 15 | 4 | 13 | 0.866666667 |
| | | OS = Mus musculus GN = Hint1 PE = 1 SV = 3 - | | | | | |
| | | [HINT1_MOUSE] | | | | | |
| 40 | O88990 | Alpha-actinin-3 OS = Mus musculus GN = Actn3 | 31 | 279 | 30 | 241 | 0.863799283 |
| | | PE = 2 SV = 1 - [ACTN3_MOUSE] | | | | | |
| 253 | Q3V1D3 | AMP deaminase 1 OS = Mus musculus | 20 | 150 | 18 | 129 | 0.86 |
| | | GN = Ampd1 PE = 1 SV = 2 - [AMPD1_MOUSE] | | | | | |
| 133 | P28650 | Adenylosuccinate synthetase isozyme 1 OS = Mus musculus | 18 | 120 | 18 | 103 | 0.858333333 |
| | | GN = Adssl1 PE = 1 SV = 2 - | | | | | |
| | | [PURA1_MOUSE] | | | | | |
| 301 | Q78ZA7 | Nucleosome assembly protein 1-like 4 OS = Mus musculus | 3 | 7 | 4 | 6 | 0.857142857 |
| | | GN = Nap1l4 PE = 1 SV = 1 - | | | | | |
| | | [NP1L4_MOUSE] | | | | | |
| 360 | Q8VCT4 | Carboxylesterase 1D OS = Mus musculus | 9 | 42 | 9 | 36 | 0.857142857 |
| | | GN = Ces1d PE = 1 SV = 1 - [CES1D_MOUSE] | | | | | |
| 409 | Q9CWJ9 | Bifunctional purine biosynthesis protein PURH | 4 | 7 | 4 | 6 | 0.857142857 |
| | | OS = Mus musculus GN = Atic PE = 1 SV = 2 - | | | | | |
| | | [PUR9_MOUSE] | | | | | |
| 393 | Q99LC5 | Electron transfer flavoprotein subunit alpha, | 9 | 39 | 7 | 33 | 0.846153846 |
| | | mitochondrial OS = Mus musculus GN = Etfa PE = 1 | | | | | |
| | | SV = 2 - [ETFA_MOUSE] | | | | | |
| 33 | O70250 | Phosphoglycerate mutase 2 OS = Mus musculus | 13 | 350 | 13 | 296 | 0.845714286 |
| | | GN = Pgam2 PE = 1 SV = 3 - [PGAM2_MOUSE] | | | | | |
| 21 | O08749 | Dihydrolipoyl dehydrogenase, mitochondrial | 10 | 45 | 9 | 38 | 0.844444444 |
| | | OS = Mus musculus GN = Dld PE = 1 SV = 2 - | | | | | |
| | | [DLDH_MOUSE] | | | | | |
| 64 | P06745 | Glucose-6-phosphate isomerase OS = Mus musculus | 23 | 311 | 22 | 262 | 0.84244373 |
| | | GN = Gpi PE = 1 SV = 4 - | | | | | |
| | | [G6PI_MOUSE] | | | | | |
| 390 | Q99KI0 | Aconitate hydratase, mitochondrial OS = Mus musculus | 27 | 207 | 25 | 174 | 0.84057971 |
| | | GN = Aco2 PE = 1 SV = 1 - | | | | | |
| | | [ACON_MOUSE] | | | | | |
| 87 | P12367 | cAMP-dependent protein kinase type II-alpha | 7 | 12 | 5 | 10 | 0.833333333 |
| | | regulatory subunit OS = Mus musculus | | | | | |
| | | GN = Prkar2a PE = 1 SV = 2 - [KAP2_MOUSE] | | | | | |
| 127 | P26516 | 26S proteasome non-ATPase regulatory subunit 7 | 3 | 6 | 3 | 5 | 0.833333333 |
| | | OS = Mus musculus GN = Psmd7 PE = 1 SV = 2 - | | | | | |
| | | [PSMD7_MOUSE] | | | | | |
| 190 | P62962 | Profilin-1 OS = Mus musculus GN = Pfn1 PE = 1 | 3 | 6 | 3 | 5 | 0.833333333 |
| | | SV = 2 - [PROF1_MOUSE] | | | | | |
| 440 | Q9DBB8 | Trans-1,2-dihydrobenzene-1,2-diol | 4 | 24 | 4 | 20 | 0.833333333 |
| | | dehydrogenase OS = Mus musculus GN = Dhdh | | | | | |
| | | PE = 1 SV = 1 - [DHDH_MOUSE] | | | | | |
| 213 | P97443 | Histone-lysine N-methyltransferase Smyd1 | 12 | 70 | 12 | 57 | 0.814285714 |
| | | OS = Mus musculus GN = Smyd1 PE = 1 SV = 3 - | | | | | |
| | | [SMYD1_MOUSE] | | | | | |
| 59 | P05201 | Aspartate aminotransferase, cytoplasmic | 14 | 122 | 14 | 99 | 0.81147541 |
| | | OS = Mus musculus GN = Got1 PE = 1 SV = 3 - | | | | | |
| | | [AATC_MOUSE] | | | | | |
| 107 | P18242 | Cathepsin D OS = Mus musculus GN = Ctsd PE = 1 | 4 | 10 | 4 | 8 | 0.8 |
| | | SV = 1 - [CATD_MOUSE] | | | | | |
| 176 | P57776 | Elongation factor 1-delta OS = Mus musculus | 2 | 5 | 2 | 4 | 0.8 |
| | | GN = Eef1d PE = 1 SV = 3 - [EF1D_MOUSE] | | | | | |
| 289 | Q64737 | Trifunctional purine biosynthetic protein | 5 | 10 | 3 | 8 | 0.8 |
| | | adenosine-3 OS = Mus musculus GN = Gart PE = 1 | | | | | |
| | | SV = 3 - [PUR2_MOUSE] | | | | | |
| 425 | Q9D1A2 | Cytosolic non-specific dipeptidase OS = Mus musculus | 3 | 5 | 2 | 4 | 0.8 |
| | | GN = Cndp2 PE = 1 SV = 1 - | | | | | |
| | | [CNDP2_MOUSE] | | | | | |
| 466 | Q9JMA1 | Ubiquitin carboxyl-terminal hydrolase 14 | 3 | 5 | 3 | 4 | 0.8 |
| | | OS = Mus musculus GN = Usp14 PE = 1 SV = 3 - | | | | | |
| | | [UBP14_MOUSE] | | | | | |
| 91 | P14152 | Malate dehydrogenase, cytoplasmic OS = Mus musculus | 9 | 114 | 8 | 90 | 0.789473684 |
| | | GN = Mdh1 PE = 1 SV = 3 - | | | | | |
| | | [MDHC_MOUSE] | | | | | |
| 267 | Q61171 | Peroxiredoxin-2 OS = Mus musculus GN = Prdx2 | 3 | 19 | 2 | 15 | 0.789473684 |
| | | PE = 1 SV = 3 - [PRDX2_MOUSE] | | | | | |
| 187 | P62897 | Cytochrome c, somatic OS = Mus musculus | 4 | 45 | 4 | 35 | 0.777777778 |
| | | GN = Cycs PE = 1 SV = 2 - [CYC_MOUSE] | | | | | |
| 34 | O70578 | Voltage-dependent calcium channel gamma-1 | 2 | 4 | 2 | 3 | 0.75 |
| | | subunit OS = Mus musculus GN = Cacng1 PE = 2 | | | | | |
| | | SV = 1 - [CCG1_MOUSE] | | | | | |
| 126 | P26443 | Glutamate dehydrogenase 1, mitochondrial | 3 | 4 | 2 | 3 | 0.75 |
| | | OS = Mus musculus GN = Glud1 PE = 1 SV = 1 - | | | | | |
| | | [DHE3_MOUSE] | | | | | |
| 149 | P45952 | Medium-chain specific acyl-CoA dehydrogenase, | 5 | 12 | 4 | 9 | 0.75 |
| | | mitochondrial OS = Mus musculus GN = Acadm | | | | | |
| | | PE = 1 SV = 1 - [ACADM_MOUSE] | | | | | |
| 324 | Q8BU30 | Isoleucine--tRNA ligase, cytoplasmic OS = Mus musculus | 2 | 4 | 2 | 3 | 0.75 |
| | | GN = Iars PE = 1 SV = 2 - | | | | | |
| | | [SYIC_MOUSE] | | | | | |
| 347 | Q8K010 | 5-oxoprolinase OS = Mus musculus GN = Oplah | 3 | 4 | 2 | 3 | 0.75 |
| | | PE = 1 SV = 1 - [OPLA_MOUSE] | | | | | |
| 422 | Q9D0K2 | Succinyl-CoA: 3-ketoacid coenzyme A transferase | 4 | 16 | 3 | 12 | 0.75 |
| | | 1, mitochondrial OS = Mus musculus GN = Oxct1 | | | | | |
| | | PE = 1 SV = 1 - [SCOT1_MOUSE] | | | | | |
| 437 | Q9DAK9 | 14 kDa phosphohistidine phosphatase OS = Mus musculus | 2 | 4 | 2 | 3 | 0.75 |
| | | GN = Phpt1 PE = 1 SV = 1 - | | | | | |
| | | [PHP14_MOUSE] | | | | | |
| 111 | P19157 | Glutathione S-transferase P 1 OS = Mus musculus | 6 | 39 | 6 | 29 | 0.743589744 |
| | | GN = Gstp1 PE = 1 SV = 2 - [GSTP1_MOUSE] | | | | | |
| 216 | P97807 | Fumarate hydratase, mitochondrial OS = Mus musculus | 12 | 58 | 10 | 43 | 0.74137931 |
| | | GN = Fh PE = 1 SV = 3 - | | | | | |
| | | [FUMH_MOUSE] | | | | | |
| 374 | Q91X72 | Hemopexin OS = Mus musculus GN = Hpx PE = 1 | 7 | 46 | 10 | 34 | 0.739130435 |
| | | SV = 2 - [HEMO_MOUSE] | | | | | |
| 141 | P32848 | Parvalbumin alpha OS = Mus musculus GN = Pvalb | 6 | 86 | 6 | 62 | 0.720930233 |
| | | PE = 1 SV = 3 - [PRVA_MOUSE] | | | | | |
| 219 | P99027 | 60S acidic ribosomal protein P2 OS = Mus musculus | 2 | 7 | 2 | 5 | 0.714285714 |
| | | GN = Rplp2 PE = 1 SV = 3 - | | | | | |
| | | [RLA2_MOUSE] | | | | | |
| 228 | Q02789 | Voltage-dependent L-type calcium channel | 4 | 7 | 4 | 5 | 0.714285714 |
| | | subunit alpha-1S OS = Mus musculus | | | | | |
| | | GN = Cacna1s PE = 1 SV = 2 - [CAC1S_MOUSE] | | | | | |
| 20 | O08709 | Peroxiredoxin-6 OS = Mus musculus GN = Prdx6 | 7 | 24 | 5 | 17 | 0.708333333 |
| | | PE = 1 SV = 3 - [PRDX6_MOUSE] | | | | | |
| 95 | P14824 | Annexin A6 OS = Mus musculus GN = Anxa6 | 20 | 88 | 18 | 62 | 0.704545455 |
| | | PE = 1 SV = 3 - [ANXA6_MOUSE] | | | | | |
| 350 | Q8K2B3 | Succinate dehydrogenase [ubiquinone] | 11 | 27 | 9 | 19 | 0.703703704 |
| | | flavoprotein subunit, mitochondrial OS = Mus musculus | | | | | |
| | | GN = Sdha PE = 1 SV = 1 - | | | | | |
| | | [SDHA_MOUSE] | | | | | |
| 61 | P05977 | Myosin light chain 1/3, skeletal muscle isoform | 12 | 274 | 12 | 192 | 0.700729927 |
| | | OS = Mus musculus GN = Myl1 PE = 1 SV = 2 - | | | | | |
| | | [MYL1_MOUSE] | | | | | |
| 388 | Q99JY0 | Trifunctional enzyme subunit beta, mitochondrial | 5 | 10 | 4 | 7 | 0.7 |
| | | OS = Mus musculus GN = Hadhb PE = 1 SV = 1 - | | | | | |
| | | [ECHB_MOUSE] | | | | | |
| 60 | P05202 | Aspartate aminotransferase, mitochondrial | 13 | 72 | 10 | 50 | 0.694444444 |
| | | OS = Mus musculus GN = Got2 PE = 1 SV = 1 - | | | | | |
| | | [AATM_MOUSE] | | | | | |
| 328 | Q8BZA9 | Fructose-2,6-bisphosphatase TIGAR OS = Mus musculus | 7 | 36 | 7 | 25 | 0.694444444 |
| | | GN = Tigar PE = 1 SV = 1 - | | | | | |
| | | [TIGAR_MOUSE] | | | | | |
| 244 | Q3TMP8 | Trimeric intracellular cation channel type A | 4 | 13 | 3 | 9 | 0.692307692 |
| | | OS = Mus musculus GN = Tmem38a PE = 1 SV = 2 - | | | | | |
| | | [TM38A_MOUSE] | | | | | |
| 50 | P02088 | Hemoglobin subunit beta-1 OS = Mus musculus | 10 | 125 | 8 | 86 | 0.688 |
| | | GN = Hbb-b1 PE = 1 SV = 2 - [HBB1_MOUSE] | | | | | |
| 114 | P20108 | Thioredoxin-dependent peroxide reductase, | 3 | 16 | 3 | 11 | 0.6875 |
| | | mitochondrial OS = Mus musculus GN = Prdx3 | | | | | |
| | | PE = 1 SV = 1 - [PRDX3_MOUSE] | | | | | |
| 84 | P11404 | Fatty acid-binding protein, heart OS = Mus musculus | 2 | 9 | 2 | 6 | 0.666666667 |
| | | GN = Fabp3 PE = 1 SV = 5 - | | | | | |
| | | [FABPH_MOUSE] | | | | | |
| 121 | P24527 | Leukotriene A-4 hydrolase OS = Mus musculus | 7 | 15 | 5 | 10 | 0.666666667 |
| | | GN = Lta4h PE = 1 SV = 4 - [LKHA4_MOUSE] | | | | | |
| 243 | Q3TMH2 | Secernin-3 OS = Mus musculus GN = Scrn3 PE = 1 | 2 | 3 | 2 | 2 | 0.666666667 |
| | | SV = 1 - [SCRN3_MOUSE] | | | | | |
| 281 | Q62407 | Striated muscle-specific serine/threonine-protein | 2 | 6 | 2 | 4 | 0.666666667 |
| | | kinase OS = Mus musculus GN = Speg PE = 1 SV = 2 - | | | | | |
| | | [SPEG_MOUSE] | | | | | |
| 346 | Q8CI51 | PDZ and LIM domain protein 5 OS = Mus musculus | 5 | 15 | 4 | 10 | 0.666666667 |
| | | GN = Pdlim5 PE = 1 SV = 4 - | | | | | |
| | | [PDLI5_MOUSE] | | | | | |
| 366 | Q8VHN7 | G-protein coupled receptor 98 OS = Mus musculus | 2 | 3 | 2 | 2 | 0.666666667 |
| | | GN = Gpr98 PE = 2 SV = 1 - [GPR98_MOUSE] | | | | | |
| 435 | Q9D8E6 | 60S ribosomal protein L4 OS = Mus musculus | 2 | 3 | 2 | 2 | 0.666666667 |
| | | GN = Rpl4 PE = 1 SV = 3 - [RL4_MOUSE] | | | | | |
| 10 | E9PZQ0 | Ryanodine receptor 1 OS = Mus musculus | 79 | 254 | 65 | 168 | 0.661417323 |
| | | GN = Ryr1 PE = 1 SV = 1 - [RYR1_MOUSE] | | | | | |
| 450 | Q9EQ20 | Methylmalonate-semialdehyde dehydrogenase | 5 | 20 | 4 | 13 | 0.65 |
| | | [acylating], mitochondrial OS = Mus musculus | | | | | |
| | | GN = Aldh6a1 PE = 1 SV = 1 - [MMSA_MOUSE] | | | | | |
| 215 | P97457 | Myosin regulatory light chain 2, skeletal muscle | 12 | 203 | 9 | 131 | 0.645320197 |
| | | isoform OS = Mus musculus GN = Mylpf PE = 1 | | | | | |
| | | SV = 3 - [MLRS_MOUSE] | | | | | |
| 271 | Q61425 | Hydroxyacyl-coenzyme A dehydrogenase, | 5 | 11 | 2 | 7 | 0.636363636 |
| | | mitochondrial OS = Mus musculus GN = Hadh | | | | | |
| | | PE = 1 SV = 2 - [HCDH_MOUSE] | | | | | |
| 175 | P56480 | ATP synthase subunit beta, mitochondrial | 25 | 318 | 23 | 201 | 0.632075472 |
| | | OS = Mus musculus GN = Atp5b PE = 1 SV = 2 - | | | | | |
| | | [ATPB_MOUSE] | | | | | |
| 277 | Q61838 | Alpha-2-macroglobulin OS = Mus musculus | 9 | 19 | 5 | 12 | 0.631578947 |
| | | GN = A2m PE = 1 SV = 3 - [A2M_MOUSE] | | | | | |
| 419 | Q9D051 | Pyruvate dehydrogenase E1 component subunit | 8 | 43 | 7 | 27 | 0.627906977 |
| | | beta, mitochondrial OS = Mus musculus GN = Pdhb | | | | | |
| | | PE = 1 SV = 1 - [ODPB_MOUSE] | | | | | |
| 489 | Q9Z1E4 | Glycogen [starch] synthase, muscle OS = Mus musculus | 17 | 102 | 19 | 64 | 0.62745098 |
| | | GN = Gys1 PE = 1 SV = 2 - | | | | | |
| | | [GYS1_MOUSE] | | | | | |
| 229 | Q03265 | ATP synthase subunit alpha, mitochondrial | 15 | 110 | 15 | 69 | 0.627272727 |
| | | OS = Mus musculus GN = Atp5a1 PE = 1 SV = 1 - | | | | | |
| | | [ATPA_MOUSE] | | | | | |
| 104 | P17563 | Selenium-binding protein 1 OS = Mus musculus | 3 | 8 | 4 | 5 | 0.625 |
| | | GN = Selenbp1 PE = 1 SV = 2 - [SBP1_MOUSE] | | | | | |
| 81 | P10639 | Thioredoxin OS = Mus musculus GN = Txn PE = 1 | 3 | 13 | 3 | 8 | 0.615384615 |
| | | SV = 3 - [THIO_MOUSE] | | | | | |
| 208 | P70695 | Fructose-1,6-bisphosphatase isozyme 2 OS = Mus musculus | 10 | 51 | 8 | 31 | 0.607843137 |
| | | GN = Fbp2 PE = 1 SV = 2 - | | | | | |
| | | [F16P2_MOUSE] | | | | | |
| 319 | Q8BMF4 | Dihydrolipoyllysine-residue acetyltransferase | 7 | 38 | 6 | 23 | 0.605263158 |
| | | component of pyruvate dehydrogenase complex, | | | | | |
| | | mitochondrial OS = Mus musculus GN = Dlat PE = 1 | | | | | |
| | | SV = 2 - [ODP2_MOUSE] | | | | | |
| 79 | P10630 | Eukaryotic initiation factor 4A-II OS = Mus musculus | 2 | 5 | 2 | 3 | 0.6 |
| | | GN = Eif4a2 PE = 1 SV = 2 - | | | | | |
| | | [IF4A2_MOUSE] | | | | | |
| 189 | P62960 | Nuclease-sensitive element-binding protein 1 | 2 | 5 | 2 | 3 | 0.6 |
| | | OS = Mus musculus GN = Ybx1 PE = 1 SV = 3 - | | | | | |
| | | [YBOX1_MOUSE] | | | | | |
| 234 | Q07417 | Short-chain specific acyl-CoA dehydrogenase, | 5 | 10 | 2 | 6 | 0.6 |
| | | mitochondrial OS = Mus musculus GN = Acads | | | | | |
| | | PE = 1 SV = 2 - [ACADS_MOUSE] | | | | | |
| 226 | Q02053 | Ubiquitin-like modifier-activating enzyme 1 | 17 | 48 | 9 | 28 | 0.583333333 |
| | | OS = Mus musculus GN = Uba1 PE = 1 SV = 1 - | | | | | |
| | | [UBA1_MOUSE] | | | | | |
| 49 | P01942 | Hemoglobin subunit alpha OS = Mus musculus | 7 | 49 | 7 | 28 | 0.571428571 |
| | | GN = Hba PE = 1 SV = 2 - [HBA_MOUSE] | | | | | |
| 142 | P35486 | Pyruvate dehydrogenase E1 component subunit | 9 | 42 | 7 | 24 | 0.571428571 |
| | | alpha, somatic form, mitochondrial OS = Mus musculus | | | | | |
| | | GN = Pdha1 PE = 1 SV = 1 - | | | | | |
| | | [ODPA_MOUSE] | | | | | |
| 315 | Q8BH64 | EH domain-containing protein 2 OS = Mus musculus | 3 | 7 | 2 | 4 | 0.571428571 |
| | | GN = Ehd2 PE = 1 SV = 1 - | | | | | |
| | | [EHD2_MOUSE] | | | | | |
| 320 | Q8BMS1 | Trifunctional enzyme subunit alpha, | 7 | 41 | 6 | 23 | 0.56097561 |
| | | mitochondrial OS = Mus musculus GN = Hadha | | | | | |
| | | PE = 1 SV = 1 - [ECHA_MOUSE] | | | | | |
| 24 | O09061 | Proteasome subunit beta type-1 OS = Mus musculus | 3 | 9 | 3 | 5 | 0.555555556 |
| | | GN = Psmb1 PE = 1 SV = 1 - | | | | | |
| | | [PSB1_MOUSE] | | | | | |
| 211 | P97355 | Spermine synthase OS = Mus musculus GN = Sms | 5 | 11 | 4 | 6 | 0.545454545 |
| | | PE = 1 SV = 1 - [SPSY_MOUSE] | | | | | |
| 183 | P61982 | 14-3-3 protein gamma OS = Mus musculus | 4 | 24 | 5 | 13 | 0.541666667 |
| | | GN = Ywhag PE = 1 SV = 2 - [1433G_MOUSE] | | | | | |
| 94 | P14602 | Heat shock protein beta-1 OS = Mus musculus | 3 | 13 | 4 | 7 | 0.538461538 |
| | | GN = Hspb1 PE = 1 SV = 3 - [HSPB1_MOUSE] | | | | | |
| 415 | Q9CZU6 | Citrate synthase, mitochondrial OS = Mus musculus | 13 | 120 | 10 | 64 | 0.533333333 |
| | | GN = Cs PE = 1 SV = 1 - [CISY_MOUSE] | | | | | |
| 471 | Q9QY76 | Vesicle-associated membrane protein-associated | 2 | 15 | 2 | 8 | 0.533333333 |
| | | protein B OS = Mus musculus GN = Vapb PE = 1 | | | | | |
| | | SV = 3 - [VAPB_MOUSE] | | | | | |
| 31 | O55126 | Protein NipSnap homolog 2 OS = Mus musculus | 4 | 17 | 3 | 9 | 0.529411765 |
| | | GN = Gbas PE = 1 SV = 1 - [NIPS2_MOUSE] | | | | | |
| 43 | P01027 | Complement C3 OS = Mus musculus GN = C3 | 6 | 17 | 4 | 9 | 0.529411765 |
| | | PE = 1 SV = 3 - [CO3_MOUSE] | | | | | |
| 70 | P07759 | Serine protease inhibitor A3K OS = Mus musculus | 3 | 21 | 2 | 11 | 0.523809524 |
| | | GN = Serpina3k PE = 1 SV = 2 - [SPA3K_MOUSE] | | | | | |
| 493 | Q9Z2I9 | Succinyl-CoA ligase [ADP-forming] subunit | 9 | 50 | 9 | 26 | 0.52 |
| | | beta, mitochondrial OS = Mus musculus | | | | | |
| | | GN = Sucla2 PE = 1 SV = 2 - [SUCB1_MOUSE] | | | | | |
| 169 | P54822 | Adenylosuccinate lyase OS = Mus musculus | 11 | 29 | 6 | 15 | 0.517241379 |
| | | GN = Adsl PE = 1 SV = 2 - [PUR8_MOUSE] | | | | | |
| 296 | Q6PIE5 | Sodium/potassium-transporting ATPase subunit | 11 | 35 | 5 | 18 | 0.514285714 |
| | | alpha-2 OS = Mus musculus GN = Atp1a2 PE = 1 | | | | | |
| | | SV = 1 - [AT1A2_MOUSE] | | | | | |
| 2 | A2AN08 | E3 ubiquitin-protein ligase UBR4 OS = Mus musculus | 4 | 4 | 2 | 2 | 0.5 |
| | | GN = Ubr4 PE = 1 SV = 1 - | | | | | |
| | | [UBR4_MOUSE] | | | | | |
| 29 | O35855 | Branched-chain-amino-acid aminotransferase, | 5 | 10 | 2 | 5 | 0.5 |
| | | mitochondrial OS = Mus musculus GN = Bcat2 | | | | | |
| | | PE = 1 SV = 2 - [BCAT2_MOUSE] | | | | | |
| 217 | P97823 | Acyl-protein thioesterase 1 OS = Mus musculus | 4 | 10 | 3 | 5 | 0.5 |
| | | GN = Lypla1 PE = 1 SV = 1 - [LYPA1_MOUSE] | | | | | |
| 237 | Q11011 | Puromycin-sensitive aminopeptidase OS = Mus musculus | 8 | 14 | 4 | 7 | 0.5 |
| | | GN = Npepps PE = 1 SV = 2 - | | | | | |
| | | [PSA_MOUSE] | | | | | |
| 447 | Q9DCW4 | Electron transfer flavoprotein subunit beta | 7 | 26 | 3 | 13 | 0.5 |
| | | OS = Mus musculus GN = Etfb PE = 1 SV = 3 - | | | | | |
| | | [ETFB_MOUSE] | | | | | |
| 468 | Q9QUR6 | Prolyl endopeptidase OS = Mus musculus | 3 | 8 | 3 | 4 | 0.5 |
| | | GN = Prep PE = 1 SV = 1 - [PPCE_MOUSE] | | | | | |
| 470 | Q9QXS1 | Plectin OS = Mus musculus GN = Plec PE = 1 SV = 3 - | 28 | 47 | 16 | 23 | 0.489361702 |
| | | [PLEC_MOUSE] | | | | | |
| 427 | Q9D2G2 | Dihydrolipoyllysine-residue succinyltransferase | 6 | 35 | 4 | 17 | 0.485714286 |
| | | component of 2-oxoglutarate dehydrogenase | | | | | |
| | | complex, mitochondrial OS = Mus musculus | | | | | |
| | | GN = Dlst PE = 1 SV = 1 - [ODO2_MOUSE] | | | | | |
| 258 | Q60597 | 2-oxoglutarate dehydrogenase, mitochondrial | 29 | 152 | 16 | 71 | 0.467105263 |
| | | OS = Mus musculus GN = Ogdh PE = 1 SV = 3 - | | | | | |
| | | [ODO1_MOUSE] | | | | | |
| 159 | P48962 | ADP/ATP translocase 1 OS = Mus musculus | 13 | 171 | 10 | 78 | 0.456140351 |
| | | GN = Slc25a4 PE = 1 SV = 4 - [ADT1_MOUSE] | | | | | |
| 171 | P55264 | Adenosine kinase OS = Mus musculus GN = Adk | 5 | 11 | 4 | 5 | 0.454545455 |
| | | PE = 1 SV = 2 - [ADK_MOUSE] | | | | | |
| 426 | Q9D1G3 | Protein-cysteine N-palmitoyltransferase HHAT- | 6 | 11 | 2 | 5 | 0.454545455 |
| | | like protein OS = Mus musculus GN = Hhatl PE = 1 | | | | | |
| | | SV = 2 - [HHATL_MOUSE] | | | | | |
| 76 | P09671 | Superoxide dismutase [Mn], mitochondrial | 4 | 20 | 4 | 9 | 0.45 |
| | | OS = Mus musculus GN = Sod2 PE = 1 SV = 3 - | | | | | |
| | | [SODM_MOUSE] | | | | | |
| 86 | P11798 | Calcium/calmodulin-dependent protein kinase | 2 | 9 | 2 | 4 | 0.444444444 |
| | | type II subunit alpha OS = Mus musculus | | | | | |
| | | GN = Camk2a PE = 1 SV = 2 - [KCC2A_MOUSE] | | | | | |
| 206 | P70404 | Isocitrate dehydrogenase [NAD] subunit gamma | 6 | 14 | 4 | 6 | 0.428571429 |
| | | 1, mitochondrial OS = Mus musculus GN = Idh3g | | | | | |
| | | PE = 1 SV = 1 - [IDHG1_MOUSE] | | | | | |
| 144 | P38647 | Stress-70 protein, mitochondrial OS = Mus musculus | 5 | 15 | 2 | 6 | 0.4 |
| | | GN = Hspa9 PE = 1 SV = 3 - | | | | | |
| | | [GRP75_MOUSE] | | | | | |
| 286 | Q64433 | 10 kDa heat shock protein, mitochondrial | 2 | 5 | 2 | 2 | 0.4 |
| | | OS = Mus musculus GN = Hspe1 PE = 1 SV = 2 - | | | | | |
| | | [CH10_MOUSE] | | | | | |
| 377 | Q91YT0 | NADH dehydrogenase [ubiquinone] flavoprotein | 6 | 15 | 3 | 6 | 0.4 |
| | | 1, mitochondrial OS = Mus musculus GN = Ndufv1 | | | | | |
| | | PE = 1 SV = 1 - [NDUV1_MOUSE] | | | | | |
| 294 | Q6P8J7 | Creatine kinase S-type, mitochondrial OS = Mus musculus | 13 | 88 | 9 | 35 | 0.397727273 |
| | | GN = Ckmt2 PE = 1 SV = 1 - | | | | | |
| | | [KCRS_MOUSE] | | | | | |
| 439 | Q9DB77 | Cytochrome b-c1 complex subunit 2, | 12 | 48 | 6 | 19 | 0.395833333 |
| | | mitochondrial OS = Mus musculus GN = Uqcrc2 | | | | | |
| | | PE = 1 SV = 1 - [QCR2_MOUSE] | | | | | |
| 314 | Q8BH59 | Calcium-binding mitochondrial carrier protein | 21 | 99 | 10 | 39 | 0.393939394 |
| | | Aralar1 OS = Mus musculus GN = Slc25a12 PE = 1 | | | | | |
| | | SV = 1 - [CMC1_MOUSE] | | | | | |
| 155 | P47934 | Carnitine O-acetyltransferase OS = Mus musculus | 10 | 23 | 4 | 9 | 0.391304348 |
| | | GN = Crat PE = 1 SV = 3 - [CACP_MOUSE] | | | | | |
| 56 | P04247 | Myoglobin OS = Mus musculus GN = Mb PE = 1 | 7 | 63 | 3 | 24 | 0.380952381 |
| | | SV = 3 - [MYG_MOUSE] | | | | | |
| 122 | P24549 | Retinal dehydrogenase 1 OS = Mus musculus | 7 | 8 | 3 | 3 | 0.375 |
| | | GN = Aldh1a1 PE = 1 SV = 5 - [AL1A1_MOUSE] | | | | | |
| 405 | Q9CQ62 | 2,4-dienoyl-CoA reductase, mitochondrial | 4 | 8 | 2 | 3 | 0.375 |
| | | OS = Mus musculus GN = Decr1 PE = 1 SV = 1 - | | | | | |
| | | [DECR_MOUSE] | | | | | |
| 481 | Q9R1P4 | Proteasome subunit alpha type-1 OS = Mus musculus | 3 | 8 | 2 | 3 | 0.375 |
| | | GN = Psma1 PE = 1 SV = 1 - | | | | | |
| | | [PSA1_MOUSE] | | | | | |
| 167 | P54071 | Isocitrate dehydrogenase [NADP], mitochondrial | 10 | 38 | 6 | 14 | 0.368421053 |
| | | OS = Mus musculus GN = Idh2 PE = 1 SV = 3 - | | | | | |
| | | [IDHP_MOUSE] | | | | | |
| 235 | Q08642 | Protein-arginine deiminase type-2 OS = Mus musculus | 16 | 64 | 6 | 22 | 0.34375 |
| | | GN = Padi2 PE = 1 SV = 2 - | | | | | |
| | | [PADI2_MOUSE] | | | | | |
| 146 | P42125 | Enoyl-CoA delta isomerase 1, mitochondrial | 5 | 15 | 2 | 5 | 0.333333333 |
| | | OS = Mus musculus GN = Eci1 PE = 1 SV = 2 - | | | | | |
| | | [ECI1_MOUSE] | | | | | |
| 264 | Q60932 | Voltage-dependent anion-selective channel | 12 | 51 | 6 | 17 | 0.333333333 |
| | | protein 1 OS = Mus musculus GN = Vdac1 PE = 1 | | | | | |
| | | SV = 3 - [VDAC1_MOUSE] | | | | | |
| 365 | Q8VEM8 | Phosphate carrier protein, mitochondrial OS = Mus musculus | 7 | 21 | 4 | 7 | 0.333333333 |
| | | GN = Slc25a3 PE = 1 SV = 1 - | | | | | |
| | | [MPCP_MOUSE] | | | | | |
| 164 | P51174 | Long-chain specific acyl-CoA dehydrogenase, | 10 | 52 | 8 | 17 | 0.326923077 |
| | | mitochondrial OS = Mus musculus GN = Acadl | | | | | |
| | | PE = 1 SV = 2 - [ACADL_MOUSE] | | | | | |
| 14 | O08528 | Hexokinase-2 OS = Mus musculus GN = Hk2 PE = 1 | 13 | 38 | 6 | 12 | 0.315789474 |
| | | SV = 1 - [HXK2_MOUSE] | | | | | |
| 232 | Q06770 | Corticosteroid-binding globulin OS = Mus musculus | 4 | 16 | 2 | 5 | 0.3125 |
| | | GN = Serpina6 PE = 1 SV = 1 - | | | | | |
| | | [CBG_MOUSE] | | | | | |
| 287 | Q64521 | Glycerol-3-phosphate dehydrogenase, | 7 | 13 | 2 | 4 | 0.307692308 |
| | | mitochondrial OS = Mus musculus GN = Gpd2 | | | | | |
| | | PE = 1 SV = 2 - [GPDM_MOUSE] | | | | | |
| 35 | O70622 | Reticulon-2 OS = Mus musculus GN = Rtn2 PE = 1 | 4 | 10 | 3 | 3 | 0.3 |
| | | SV = 1 - [RTN2_MOUSE] | | | | | |
| 184 | P62141 | Serine/threonine-protein phosphatase PP1-beta | 6 | 14 | 3 | 4 | 0.285714286 |
| | | catalytic subunit OS = Mus musculus GN = Ppp1cb | | | | | |
| | | PE = 1 SV = 3 - [PP1B_MOUSE] | | | | | |
| 292 | Q6P5E4 | UDP-glucose: glycoprotein glucosyltransferase 1 | 4 | 7 | 2 | 2 | 0.285714286 |
| | | OS = Mus musculus GN = Uggt1 PE = 1 SV = 4 - | | | | | |
| | | [UGGG1_MOUSE] | | | | | |
| 263 | Q60931 | Voltage-dependent anion-selective channel | 5 | 23 | 2 | 6 | 0.260869565 |
| | | protein 3 OS = Mus musculus GN = Vdac3 PE = 1 | | | | | |
| | | SV = 1 - [VDAC3_MOUSE] | | | | | |
| 411 | Q9CZ13 | Cytochrome b-c1 complex subunit 1, | 9 | 24 | 4 | 6 | 0.25 |
| | | mitochondrial OS = Mus musculus GN = Uqcrc1 | | | | | |
| | | PE = 1 SV = 2 - [QCR1_MOUSE] | | | | | |
| 194 | P63038 | 60 kDa heat shock protein, mitochondrial | 8 | 18 | 3 | 4 | 0.222222222 |
| | | OS = Mus musculus GN = Hspd1 PE = 1 SV = 1 - | | | | | |
| | | [CH60_MOUSE] | | | | | |
| 214 | P97447 | Four and a half LIM domains protein 1 OS = Mus musculus | 6 | 18 | 3 | 4 | 0.222222222 |
| | | GN = Fhl1 PE = 1 SV = 3 - | | | | | |
| | | [FHL1_MOUSE] | | | | | |
| 163 | P50544 | Very long-chain specific acyl-CoA | 7 | 19 | 3 | 4 | 0.210526316 |
| | | dehydrogenase, mitochondrial OS = Mus musculus | | | | | |
| | | GN = Acadvl PE = 1 SV = 3 - [ACADV_MOUSE] | | | | | |
| 151 | P47738 | Aldehyde dehydrogenase, mitochondrial | 6 | 15 | 3 | 3 | 0.2 |
| | | OS = Mus musculus GN = Aldh2 PE = 1 SV = 1 - | | | | | |
| | | [ALDH2_MOUSE] | | | | | |
| 46 | P01864 | Ig gamma-2A chain C region secreted form | 3 | 12 | 2 | 2 | 0.166666667 |
| | | OS = Mus musculus PE = 1 SV = 1 - | | | | | |
| | | [GCAB_MOUSE] | | | | | |
| 119 | P23927 | Alpha-crystallin B chain OS = Mus musculus | 4 | 13 | 2 | 2 | 0.153846154 |
| | | GN = Cryab PE = 1 SV = 2 - [CRYAB_MOUSE] | | | | | |
| 369 | Q91VD9 | NADH-ubiquinone oxidoreductase 75 kDa | 9 | 26 | 3 | 4 | 0.153846154 |
| | | subunit, mitochondrial OS = Mus musculus | | | | | |
| | | GN = Ndufs1 PE = 1 SV = 2 - [NDUS1_MOUSE] | | | | | |
| 28 | O35350 | Calpain-1 catalytic subunit OS = Mus musculus | 7 | 15 | 2 | 2 | 0.133333333 |
| | | GN = Capn1 PE = 1 SV = 1 - [CAN1_MOUSE] | | | | | |
| 444 | Q9DC69 | NADH dehydrogenase [ubiquinone] 1 alpha | 7 | 24 | 2 | 2 | 0.083333333 |
| | | subcomplex subunit 9, mitochondrial OS = Mus musculus | | | | | |
| | | GN = Ndufa9 PE = 1 SV = 2 - | | | | | |
| | | [NDUA9_MOUSE] | | | | | |

What is claimed is:

1. A method for preventing or treating muscle loss in a subject in need thereof, comprising:
administering to the subject an effective amount of at least one indoleamine 2,3-dioxygenase (“IDO”) inhibitor to stop or reverse the progression of muscle loss in the subject.

2. The method of claim 1, wherein the at least one IDO inhibitor is 1-methyl-D-tryptophan.

3. The method of claim 1, wherein the subject has or is susceptible of developing sarcopenia.

4. The method of claim 1, wherein the at least one IDO inhibitor is administered to the subject in an effective amount of about 200 to about 2500 mg/kg body weight.

5. A method for preventing or treating sarcopenia in a subject in need thereof, comprising:
administering to the subject a therapeutically effective amount of a pharmaceutical composition comprising an effective amount of at least one IDO inhibitor and a pharmaceutically acceptable excipient to treat or prevent sarcopenia.

6. The method of claim 5, wherein the at least one IDO inhibitor is 1-methyl-D-tryptophan.

7. The method of claim 5, wherein the subject has or is susceptible of developing sarcopenia.

8. The method of claim 5, wherein the pharmaceutical composition is formulated for oral delivery.

9. The method of claim 5, wherein the pharmaceutical composition is formulated as an extended release formulation.

10. The method of claim 5, wherein the pharmaceutical composition is administered to the subject in a therapeutically effective amount of about 200 to about 2500 mg/kg body weight.

11. A method for maintaining or increasing muscle mass and/or muscle strength in a subject in need thereof, comprising:
administering to the subject an effective amount of at least one IDO inhibitor to increase muscle mass and/or muscle strength in the subject.

12. The method of claim 11, wherein the subject has or is susceptible of developing sarcopenia.

13. The method of claim 11, wherein the at least one IDO inhibitor is 1-methyl-D-tryptophan, 1-methyl-L-tryptophan, methylthiohydantoin-dl-tryptophan, or any combination thereof.

14. The method of claim 13, wherein the at least one IDO inhibitor is 1-methyl-D-tryptophan.

15. The method of claim 11, wherein the muscle mass and/or muscle strength of the subject is increased by at least 10 percent when compared to levels of muscle mass and/or muscle strength prior to administration.
